(19) United States
(12) Patent Application Publication
(10) Pub. No.: US 2021/0291383 A1
(43) Pub. Date: Sep. 23, 2021

(54) SOFT ROBOTIC MANIPULATOR

(71) Applicant: IMPERIAL COLLEGE OF SCIENCE, TECHNOLOGY AND MEDICINE, London (GB)

(72) Inventors: Ferdinando Rodriguezy BAENA, London (GB); Arnau Garriga CASANOVAS, Molins de Rei (ES)

(21) Appl. No.: 17/260,511

(22) PCT Filed: Jul. 29, 2019
(86) PCT No.: PCT/GB19/52116

§ 371 Date: Jan. 14, 2021

(30) Foreign Application Priority Data
Jul. 30, 2018 (GB) 1812408.1

(51) Int. Cl. B25J 15/00; B25J 19/00; B25J 15/12

(52) U.S. Cl. CPC B25J 15/0023; B25J 15/12; B25J 19/007

(57) ABSTRACT

A soft robotic manipulator adapted to be activated by a pressurised fluid having a first end, a second end, an outer wall and an axis, and comprising a plurality of segments extending co-axially along the manipulator, such that the outer wall of each segment forms part of the outer wall of the manipulator, each segment having a first end and a second end and an outer wall and further comprising a plurality of chambers contained within the outer wall, each of which chambers extends from the first end to the second end, wherein each manipulator segment further comprises a central element extending along the axis of the manipulator segment, and a plurality of partition walls extending from the central element to the outer wall, the chambers being defined by the partition walls and the outer wall, wherein the outer wall of the manipulator comprises the outer wall of each segment.

This invention relates to a soft robotic manipulator, and to a method of designing a soft robotic manipulator. The invention relates particularly to soft robotic manipulators that are activated by a pressurised fluid.

Soft robots are commonly defined as devices primarily composed of low stiffness material which are frequently used to achieve significant displacements by means of structural deformation. In order to achieve the significant displacements by means of structural deformation, suitable materials for forming soft robots are generally capable of undergoing strains of the order of 100% up to 1000%. A range of different low stiffness materials may be used, and typically the materials will be, for example rubbers with Young's moduli in the range of 10⁴ to 10⁹. There have been many developments in the field of soft robotics in recent years with devices aimed at a range of applications being developed. These applications include minimally invasive surgery, micro-gripping and swimming.

Pressurized fluids are the most common and therefore relevant means of actuation in soft robotics. Soft robots with fluidic actuation are generally used either as manipulators, as limbs for locomotion, or as actuators in more complex systems such as rehabilitation or assistive devices.

The majority of these applications require the soft robot to provide a controlled motion between two points of interest in a solid structure while supporting external forces and moments.

Soft robotic manipulators with fluidic actuation are devices with easily deformable structures that comprise a set of chambers that can be pressurised to achieve structural deflection. Such soft robotic manipulators can be classified according to the motion they provide between two points of interest between which controlled motion is required. The motion is provided by pressurising the soft robotic device.

This results in three categories of robotic manipulators; devices that provide elongation; contraction; and bending.

The design of devices that provide bending, is challenging, and a general rationale for their design is not available. A problem therefore existing with known soft robotic devices categorised as bending devices is that the design of such devices generally takes place using intuition, rather than by following a design process.

According to a first aspect of the present invention there is provided a soft robotic manipulator adapted to be activated by a pressurised fluid having a first end, a second end, an outer wall and an axis, and comprising a plurality of segments extending co-axially along the manipulator, such that the outer wall of each segment forms part of the outer wall of the manipulator, each segment having a first end and a second end and an outer wall and further comprising a plurality of chambers contained within the outer wall, each of which chambers extends from the first end to the second end, wherein each manipulator segment further comprises a central element extending along the axis of the manipulator segment, and a plurality of partition walls extending from the central element to the outer wall, the chambers being defined by the partition walls and the outer wall, wherein the outer wall of the manipulator comprises the outer wall of each segment.

The soft robotic manipulator is thus formed from a plurality of manipulator segments which extend axially along the length of the manipulator and are coaxial with the manipulator. The outer wall of the manipulator is made up of the outer walls of each of the segments.

A plurality of segments may be stacked serially in order to create the full manipulator.

Each manipulator segment is designed to bend and each segment will have one or two degrees of freedom (DOF).

A typical soft robotic manipulator according to embodiments of the first aspect of the invention may be made up of three manipulator segments each of which segments having two degrees of freedom. Such an arrangement will lead to a manipulator with six degrees of freedom.

Each manipulator segment may have any convenient number of chambers, but in embodiments of the invention each manipulator segment comprises three chambers. The chambers extend axially between the first and second ends of each manipulator segment.

In embodiment of the invention the outer wall has a substantially circular cross-section. This means that in such embodiments of the invention the manipulator will be substantially cylindrical when the manipulator is in a non-deployed state.

Each manipulator segment is adapted to bend and move such that the first end moves relative to the second end. This means that the initial cylindrical shape of the manipulator will change during use of the manipulator although the manipulator will remain substantially tubular during use.

In other embodiments of the invention, the outer wall has a substantially rectangular cross-section.

In embodiments of the invention, the central element comprises a rod. In embodiments of the invention, the rod is an inextendible rod.

In embodiments of the invention, the central element comprises a sheet, and in embodiments of the invention the sheet is an inextendible sheet.

In such embodiments of the invention, the partition walls may have low stiffness in order to facilitate cross-section deformation, and the outer wall may be made of a low stiffness material. In embodiments of the invention, the partition walls will be formed from a material having a Young's modulus in the range of 10⁴ to 10⁹.

In embodiments of the invention the outer wall is made from a material having a Young's modulus in the range of 10⁴ to 10⁹.

In some embodiments of the invention the partition walls are made of the same material as the outer wall. In other embodiments of the invention the partition walls are made from a material that is different to material used to make the outer wall.

The partition walls and the outer wall may be made from a silicone based material or a rubber. In embodiments of the invention the outer wall and the partition walls are made from a rubber with a Shore hardness between OO30 and OO90, using the Shore OO scale, or similarly between A0 and A60 using the Shore A scale.

In embodiments of the invention, the outer wall has a thickness of between ¼ and 1/20 of the total diameter of the manipulator.

In such embodiments of the invention, the outer wall and the partitions walls may be made of the same material.

The combination of the low stiffness of each segment of the manipulator together with the thickness of the outer wall enables the manipulator to bend with low resistance.

In embodiments of the invention where the partition walls are made of a different material to the material used for the outer wall, the stiffness of the partition walls may be expressed relative to the stiffness of the outer wall. In such embodiments of the invention, the outer wall may be made from materials with a Shore hardness of between OO30 and OO90, using the Shore OO scale, or similarly between A0 and A60 using the Shore A scale. In such embodiments of the invention the partition walls may have a stiffness between ⅓ of the stiffness of the outer wall and twice the stiffness of the outer wall.

In embodiments of the invention, the outer wall comprises fibre and rubber.

The diameter of the outer wall can vary depending on the use to which the manipulator is to be put.

In some embodiments of the invention, the diameter of the outer wall will be approximately 6 mm. In such an embodiment of the invention the length of the manipulator may be approximately 30 mm. A manipulator having dimensions of this order is suitable for use in minimal invasive surgery applications.

In such embodiments of the invention, the outer wall may have a thickness of approximately 0.4 mm and the partition walls may also have a thickness of 0.4 mm.

In embodiments of the invention the outer wall comprises a tubular structure having notches. Optionally, the tubular structure is formed from a metallic material. In embodiments of the invention the metallic material is nitinol.

In such embodiments of the invention are able to bend with minimal resistance whilst support compression forces.

In embodiments of the invention, the outer wall has a pleated structure. Such a structure can be regarded as similar to an accordion. This enables the wall to support compression forces whilst minimising resistance to bending.

In other embodiments of the invention the rod may be extendible. In such embodiments of the invention the rod may be made from a material having a stiffness equivalent to that of a fibre with 0.1 mm diameter and a Young's modulus between 1e⁷ to 1e¹⁰.

An advantage of having such a central element is that the tension created by the central element does not introduce compression forces on the outer wall that cause buckling.

In embodiments of the invention the manipulator have a central element which is inextendible and is made of parts with negligible bending stiffness and rigid parts, whereby when a differential pressure is applied in the chambers, the increase in volume in the pressurised chambers is maximised for a given increase in bending, and thus the force of the device is maximised.

In embodiments of the invention the total cross-section of the device occupies all available space in a selected application.

In embodiments of the invention the central element acts as a stiff wall in an extending device, and as a protruding wall in a contracting device.

This means that the resultant device combines extending and contracting operation into a fully integrated device.

According to a second aspect of the present invention there is provided a method of designing a soft robotic manipulator with fluidic actuation comprising the steps of;
i. identifying the conditions under which the manipulator will be operated
ii. identifying the requirements to be fulfilled by the manipulator;
iii. based on i and ii, selecting an extending, contracting or combination type manipulator;
iv. depending on the category of manipulator selected, and the conditions and requirements to be met, create a preliminary cross-section design;
v. then consider the optimal stiffness design required in light of ii, iii, and iv;
vi. repeat steps iv and v as necessary in order to optimize the design;
vii. identify the layout of the manipulator
viii. optimize the design.

By means of the present invention, a general method may be followed in order to enable a soft robotic manipulator to be designed for a range of applications. By means of the present invention therefore it will no longer be necessary to use an intuitive approach to the design of such soft robotic manipulators. This means that the design process is rationalised and outlined to be methodical, and an appropriate design of soft robotic manipulator is arrived at that will provide improved results for the application in question.

Step i. comprises the step of identifying the conditions under which the manipulator will be operated. in embodiments of the invention this comprises determining the spatial constraints existing in the environment in which the soft robotic manipulator is to be operated.

Step ii. Involves the identification of the requirements to be fulfilled by the manipulator. In embodiments of the invention this comprises identifying a desired deflection of the manipulator claims operation of the manipulator.

Step iii, comprises the step of selecting and extending, contracting all combination type manipulator based on the conditions identified in Steps i. and ii. The manipulator may thus be considered to fall within one of these three categories.

In embodiments of the invention it may not be possible at this stage to choose between the extending and contracting categories. In such circumstances, therefore Step iii. will result in both an extending and a contracting device being chosen for the design process.

In such embodiments of the invention the design process will be carried out for both an extending device and a contracting device, and at the end of the design process, the most appropriate device will be chosen.

In Step iv. once the category of device has been chosen, the total cross section available for the manipulator to occupy will be determined. Generally, the manipulator should occupy all room available at a desired deflection.

In some cases, braids or braces may be introduced in order to adapt the total cross section to the spatial constraints pertaining.

Based on all of these factors, a preliminary cross sectional design may be determined. Such a design will consider the cross sectional area and shape of the manipulator together with the thickness of the walls defining the manipulator.

Step v. involves determining the optimal stiffness design required. In embodiments of the invention, Step v. comprises the step of determining a stiffness distribution of the robotic manipulator.

Next, in accordance with Step vi., Steps iv. and v. are repeated as necessary in order to optimise the design.

In accordance with Step vii., the most suitable design layout for a soft robotic manipulator may be determined. In some cases methods according to embodiments of the invention may show that a compromise is necessary, as not all design principles can be concurrently satisfied.

Finally, in accordance with Step viii. of the methods according to the invention, the design is optimised.

In embodiments of the invention the value of certain design parameters may need to be optimised. In embodiments of the invention, finite element (Fₑ) simulations can be used to optimise the parameters and resolve the compromises, yielding the final design. The Fₑ simulations can also be used to compare final performance of the designs in a case that both an extending and a contracting device have been explored. The better of the two devices can then be selected at this stage.

The invention will now be further described by way of example only with reference to the accompanying drawings in which:

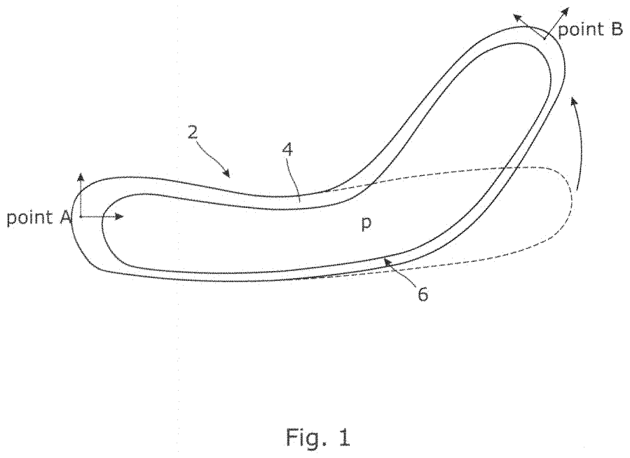

FIG. 1 is schematic representation of a generic bending device linking two points of interest A and B;

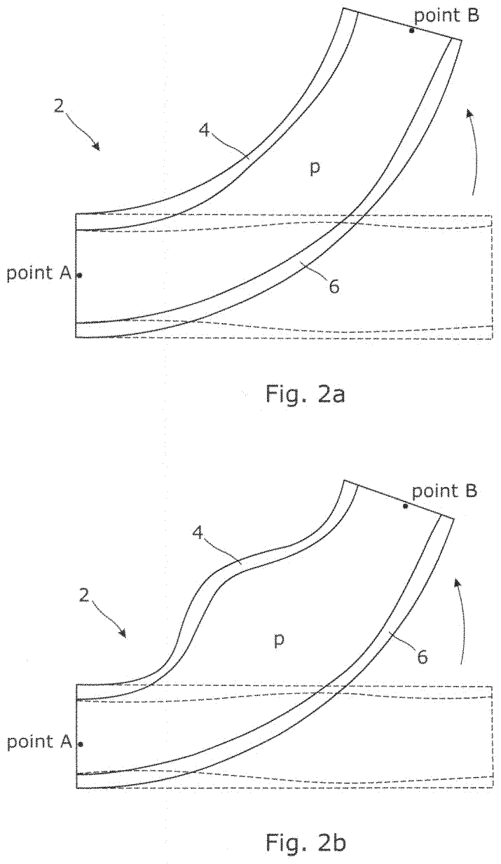

FIGS. 2a and 2b are schematic representations of a extending type device and contracting type device respectively;

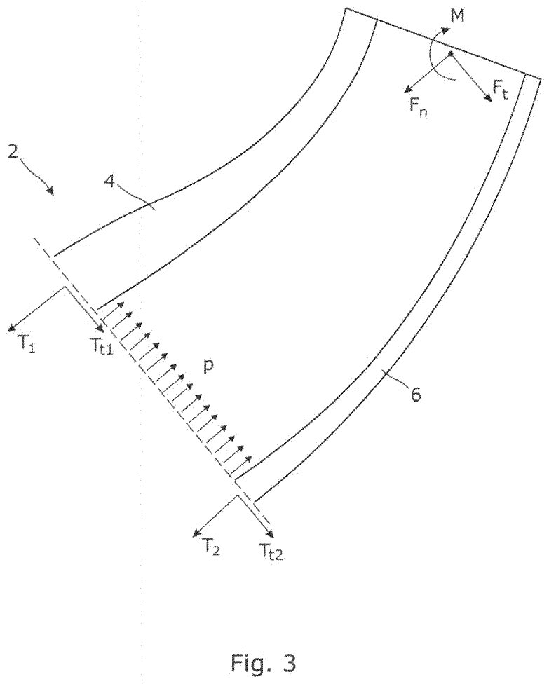

FIG. 3 is a schematic representation of the device 2 isolated at an arbitrary cross section perpendicular to the vector between the two points of interest A and B;

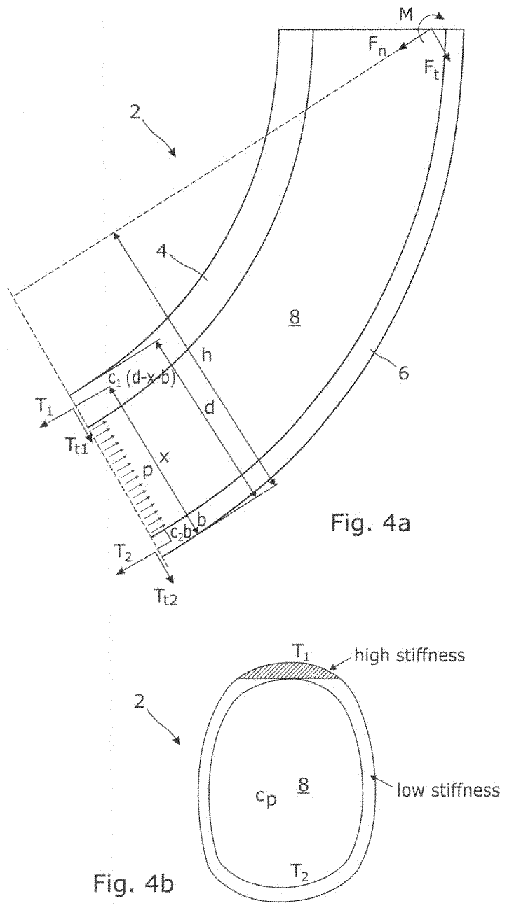

FIG. 4a is an equilibrium diagram of an extending device isolated at an arbitrary cross section exposing the reaction forces, aggregated into T₁ and T₂, and the pressure applied to the fluid;

FIG. 4 is a cross sectional representation of a 3D device with variable stiffness;

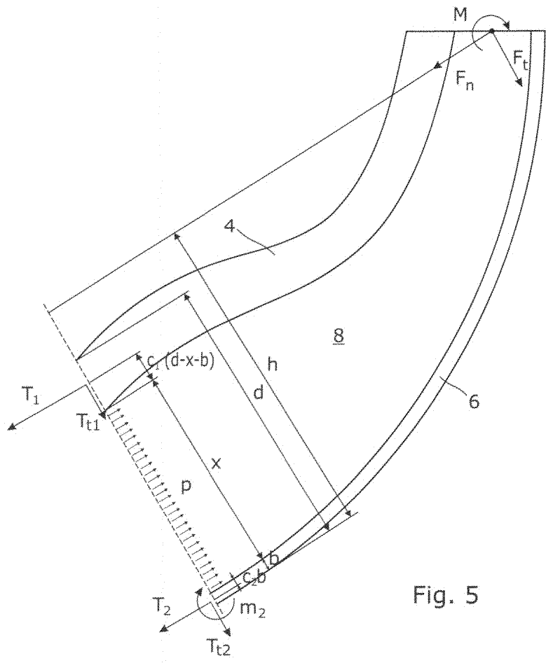

FIG. 5 is a equilibrium diagram of a 2D contracting device isolated at an arbitrary cross section exposing reaction forces as well as pressure;

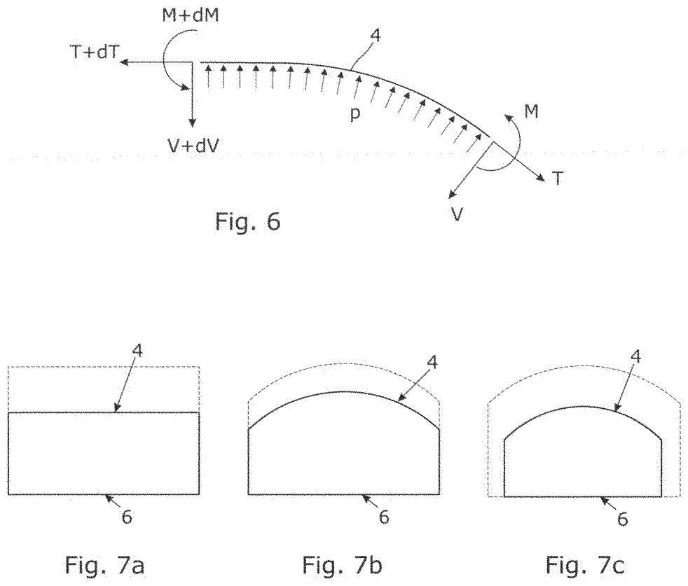

FIG. 6 is a schematic diagram showing the equilibrium of a differential wall element;

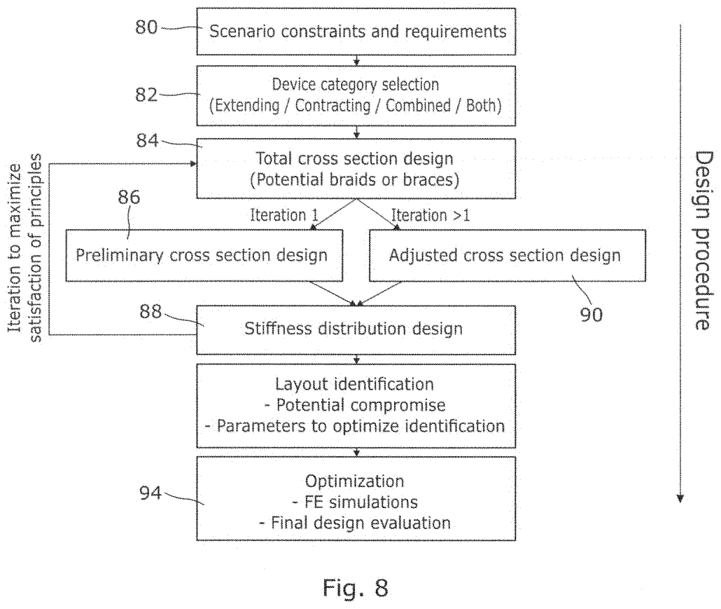

FIGS. 7a to 7c are schematic representations of a typical cross sectional design in a scenario with rectangular constraints, in a general scenario with curved constraints, and a undesirable cross sectional design in a general scenario respectively;

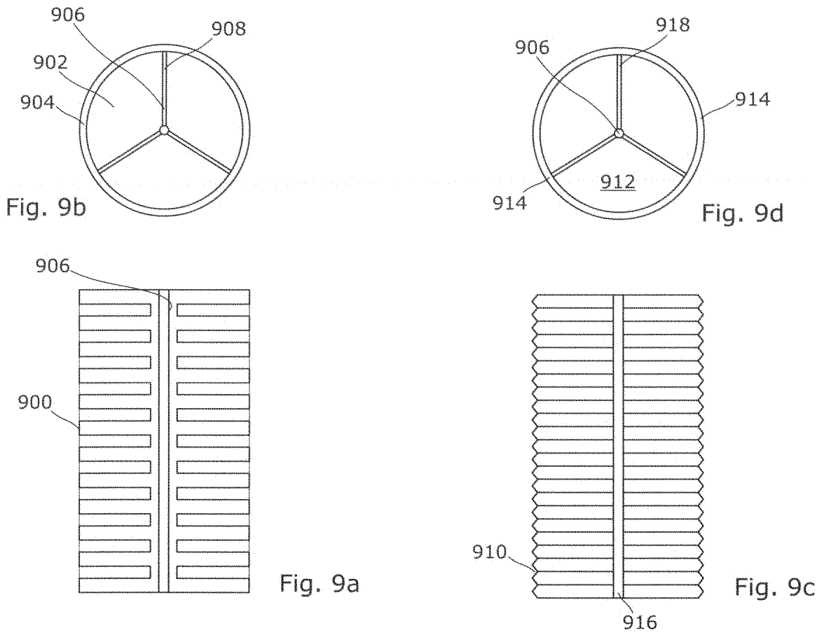

FIG. 8 is a schematic representation outlining a method for designing a soft robotic manipulator according to embodiments of the invention;

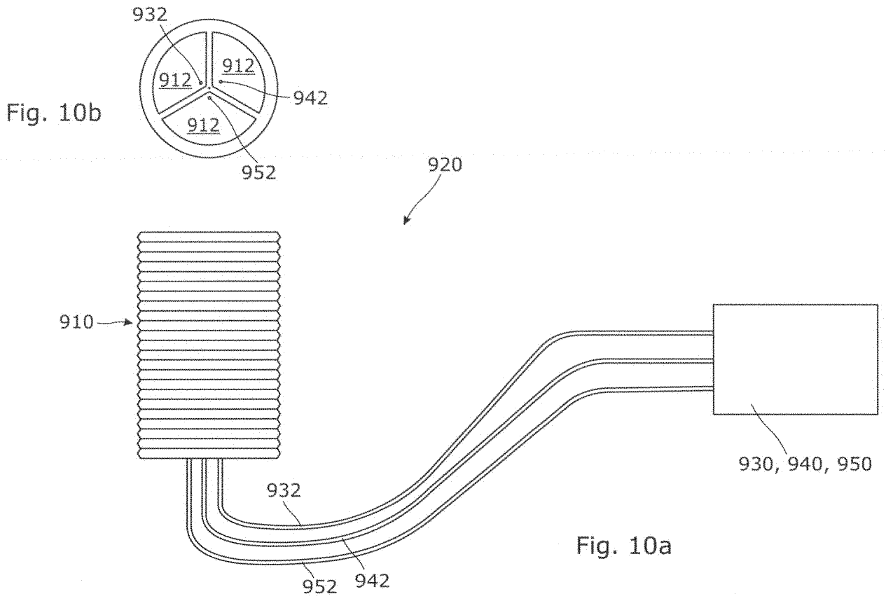

FIGS. 9a and 9b are schematic representations of an embodiment of a manipulator according to the first aspect of the invention for use in minimal invasive surgery;

FIGS. 9c and 9d are schematic representations of another embodiment of an invention suitable for use in minimal invasive surgery;

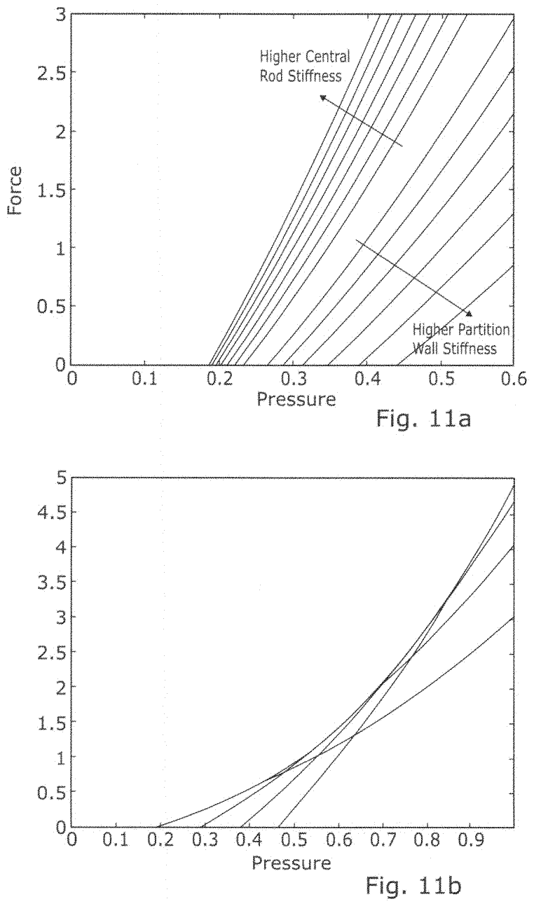

FIGS. 10a and 10b are schematic representations of a system incorporating the manipulator of FIGS. 9c and 9d;

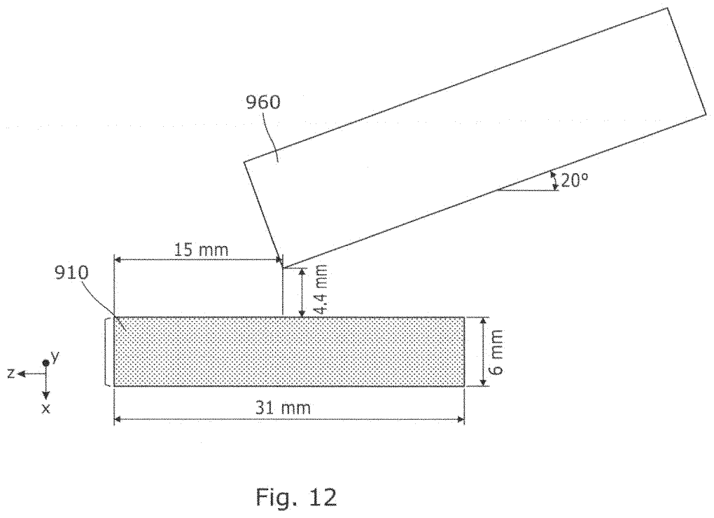

FIG. 11a is a qualitative graph illustrating the design trends corresponding to the variation of LCRS in a manipulator where the remainder of the design remains equal, and the variation of PWS, where the rest of the design remains equal;

FIG. 11b is a qualitative graph illustrating the performance of designs optimised for different ρₘₐₓ in terms of their PWS of LCRS;

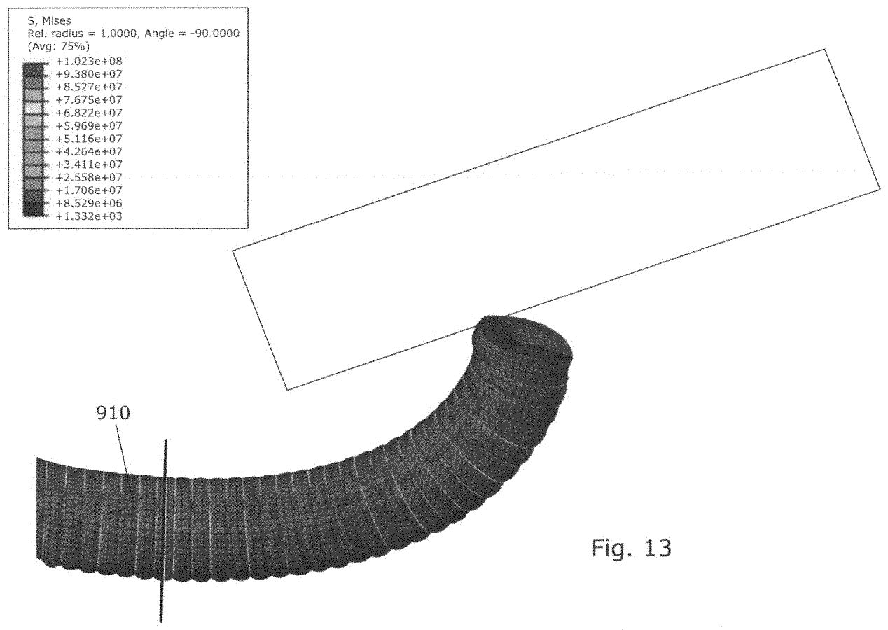

FIG. 12 is a schematic representation of a simulation with a soft robotic manipulator in rigid block;

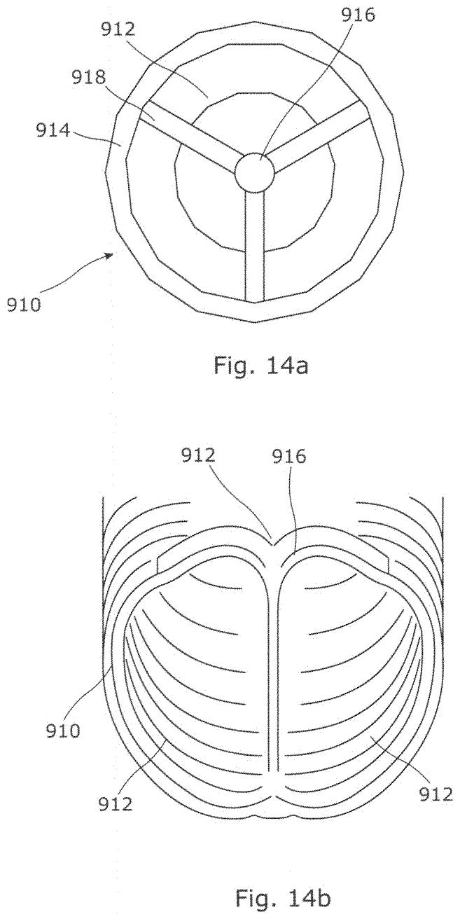

FIG. 13 is a schematic representation showing the simulation of deformed geometry in a manipulator made according to embodiments of the invention;

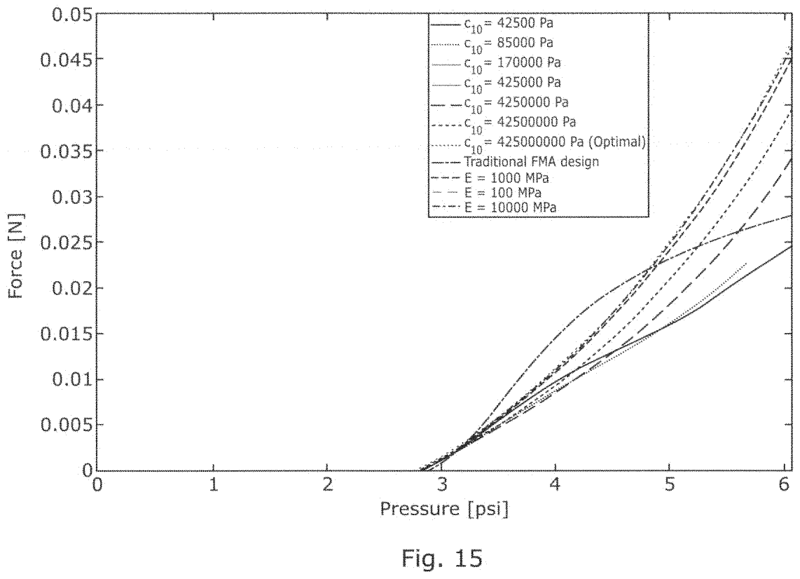

FIGS. 14a and 14b are schematic representations of a cross-sectional representation of an embodiment of the invention showing deformation of one of the chambers;

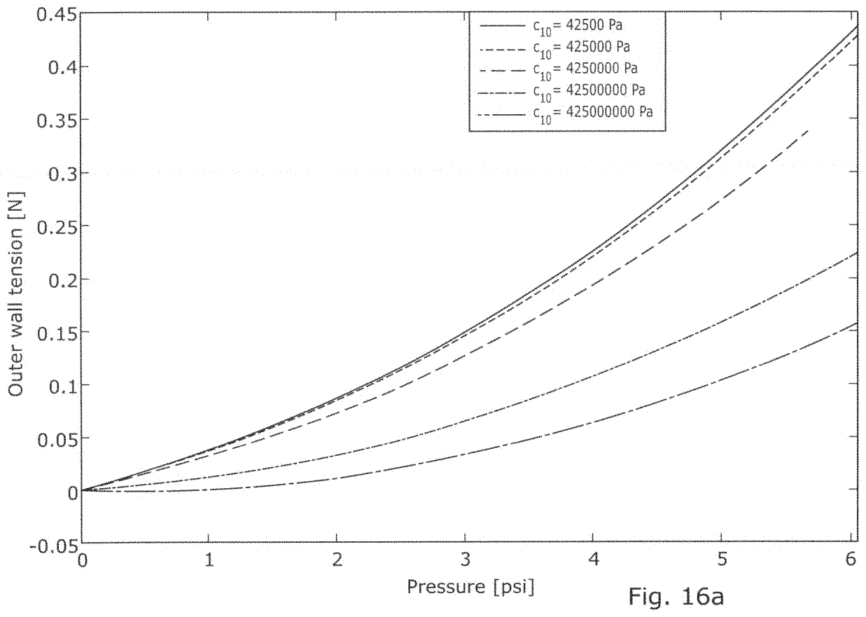

FIG. 15 is a schematic representation showing lateral force as a function of pressure for four different designs; and

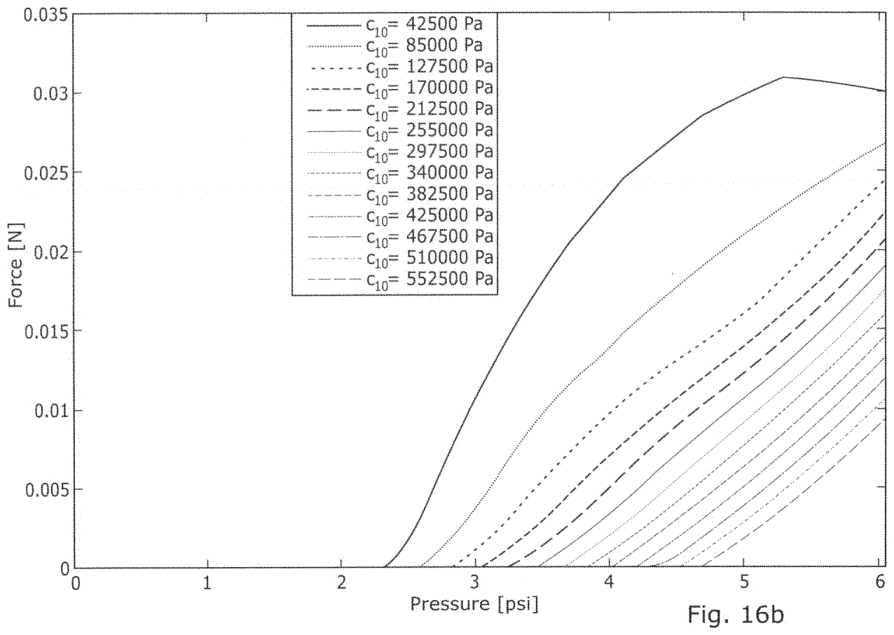

FIGS. 16a and 16b are schematic representations showing the outer wall tension as a function of pressure and the lateral forces as a function of pressure.

The purpose of the devices considered here is to provide a desired motion between two points on the device, which in this case is associated with bending, together with a certain force. In soft robots with fluidic actuation, the motion is achieved by pressurising a set of chambers in the device to produce structural deformation. The most common scenario of interest it that where the robot must generate work to produce the motion, overcoming external forces and moments.

The design problem is to select the geometry and structural properties of the soft robot to achieve the desired motion and maximize a specified performance. The design problem considered is completely general, without predefined design variable. Solving this problem generally requires determining the solution to a non-linear structural problem with large deformations, for which analytical solutions are not available in general.

Thus, in accordance with present invention an innovative approach to the design of soft robotic manipulators is provided.

The maximum pressure that a soft robot design can withstand can be very complex to determine, hindering subsequent design optimization. Frequently, however, the pressure limit is primarily dictated by the sealing points in the chambers. In addition, in the common case of medical applications, the maximum pressure can also be limited to guarantee the safety of the patient during a malfunction of the device.

In situations where the sealing points are highly resistant, and the application does not restrict the pressure values employed, the maximum pressure depends on the design of the device, and in particularly is strongly affected by the thickness of any outer walls of the device.

In “real life” situations therefore the maximum pressure will be determined by the operating conditions of the manipulator together with the physical properties of the manipulator.

The performance criteria for the optimization must be related to the purpose of these devices, i.e. to provide a bending motion while supporting external forces and moments. Typically, soft robotic manipulators are required to be capable of reaching a specific deflection determined by the desired workspace. The forces and moments they can support at that deflection tend to be their main limitation. In this regard, the optimization objective selected in this work is to maximise the forces and moments that can be supported while achieving a desired deflection, and with a given maximum pressure.

The forces and moments that soft robotic manipulators can support depend on the maximum pressure they can withstand, with higher pressures enabling higher forces and moments. However, as previously noted, in frequent cases the maximum pressure can be limited by the application or by the sealing points. In other cases, the maximum pressure can be increased with the selection of design, but that typically requires the use of thicker outer walls, which introduce bending stiffness and occupy room in the cross section, and can thus reduce performance, as elucidated in the analysis presented in the following sections. Hence, in many cases, the maximum pressure is either given or limited, and this represents the most relevant design case. The case with maximum pressure determined by the design selection is a direct extension of the case with given maximum pressure, simply adding the selection of the outer wall design (mostly in terms of thickness) to the problem.

The study and derivation of the design of manipulators is then developed in the following first for the common case of given maximum pressure, for which design principles are extracted. In this case, the optimization objective is to maximise the forces and moments that can be supported while achieving a desired deflection, and with a given maximum pressure. Afterwards, the analysis and derivation of the design are extended to a case where the maximum pressure is not limited by the scenario nor the sealing points, and instead is determined by the outer wall thickness.

Any potential design must consist of a general structure 2 linking the two points of interest as illustrated in FIG. 1 in which the device links points A and B. In soft robots with fluidic actuation, the structure is passive, and therefore the design must contain a set of chambers that can be pressurised to generate the designed motion by deforming the structure. This set of chambers must generally cover the region between the two points of interest in a nearly continuous manner.

The set of chambers, together with the direction of bending, which is approximately perpendicular to the vector between the two points of interest, define two sides of the device, which can be considered as two walls 4, 6. Kinematic considerations show that, in order to achieve bending, a differential deformation in the structure at either side of the device is required. This involves either one wall extending more than the other, or one wall contracting more than the other. Soft robotic manipulators can therefore generate bending in two elementary ways, and the designs can be classified accordingly, leading to two general categories: extending-type devices, and contracting-type devices, as illustrated in FIGS. 2a and 2b.

The equilibrium of a system corresponding to the general design isolated at an arbitrary cross section perpendicular to the vector between the two points of interest can then be considered, as shown in FIG. 3. This exposes the reaction forces as well as the pressure applied by the fluid. The system equilibrium can thus be used to provide insight into the mechanical behaviour and to study the design. Before a detailed study, the equilibrium can first be applied to the two categories of soft robotic manipulators, extending and contracting devices, to outline the design layouts, as described in the following two paragraphs.

Considering the equilibrium in extending devices, this indicates that the pressure in the chambers generally creates tensioning reactions on the structure. The reactions associated to each side of the structure depend on the design. These reactions translate into deformations, with the elongation of each side depending on the stiffness in the longitudinal direction. The differential elongation necessary for bending can therefore be achieved with either an asymmetric pressure loading or an asymmetric longitudinal stiffness. It should be noted that the reactions can also produce lateral expansion, but this generally does not contribute to elongation, rather the opposite, so it is undesirable in extending devices. Thus, the layout of extending devices must consist of an elongated structure that cannot expand radially and has a combination of asymmetric geometry and asymmetric longitudinal stiffness so that one side extends more than the other.

Considering the equilibrium in contracting devices, this also shows that the pressure generates tensioning reactions. Contraction of a region of the structure can therefore not be achieved with a compression in that region, and instead one side of the structure must either protrude outwards or buckle inwards. The layout of the contracting devices must then consist of a structure with one side that either protrudes or buckles to produce a contraction while the other side maintains the original length, resulting in bending. The equilibrium analysis indicates that both the design geometry and the longitudinal and bending stiffnesses affect the reaction forces, the protrusion geometry, and ultimately the performance.

Considering that extending and contracting devices are the only alternatives to produce bending, the study of these two layouts represents a complete study of the design of soft robotic manipulators with fluidic actuation. Devices combining extension and contraction are also possible, and their design is a combination of the design principles for both types of operation.

Extending devices achieve bending thanks to a differential extension in their structure when pressurised, which is created by a design asymmetry in terms of geometry and stiffness. The design of extending devices is studied in detail in this section in order to derive a set of design principles and determine the design that maximises the design objective.

The design objective in the derivation set out below referring to extending and contracting devices is to achieve a desired deflection and maximise the force for a given maximum pressure. The method according to embodiments of the invention focusses on the design to maximise the forces and moments that can be supported at a given deflection with a given pressure. The method also considers the design objective of reaching the desired deflection with a minimum pressure. These results are combined to extract design principles and determine the most suitable design and the overall analysis is finally generalised to 3D.

Design of Extending Devices

Equilibrium Approach

Equilibrium Formulation

The equilibrium of an extending device 2 isolated at an arbitrary cross section can be considered, as shown in FIG. 4a, exposing the reactions as well as the pressure applied by the fluid. The equilibrium of moments and forces in the direction perpendicular to the cross section can thus be imposed as

$${T}_{1} + {T}_{2} = px - {F}_{n} \\ {T}_{1} [ {c}_{1} d + x ( 1 - {c}_{1} ) + b ( {c}_{2} - {c}_{1} ) ] - p \frac{{x}^{2}}{2} - {pxc}_{2} b + {F}_{n} ( h - b ( 1 - {c}_{2} ) ) = M \qquad ( 1 )$$

where d denotes the total region of the cross section, x represents the region of the cross section corresponding to the pressurized fluid in pressurized chamber 8, and b is the region of the cross section corresponding to wall 6. The external forces are decomposed into two directions, parallel and perpendicular to the cross section. The perpendicular forces are aggregated into a resulting normal force, denoted by Fₙ, and the parallel forces are aggregated into a resulting tangential force Fₜ. RA corresponds to the sum of external moments together with the moment created by Fₜ with respect to the cross section.

The distributed normal stresses corresponding to wall 4 and 6 are aggregated into two equivalent forces, denoted by T₁ and T₂, respectively, while the distributed tangential stresses are aggregated into Tₜ₁ and Tₜ₂, respectively. The location of the equivalent line of application of T₁ and T₂ is defined by the non-dimensional parameters c₁ and c₂, respectively. The specific equivalent line of application of these two forces may not be constant and can be difficult to determine as it depends on the specific stress distribution, which is determined by a complex structural behavior. However, considering that, in soft robots with fluidic actuation, and particularly in extending devices, the walls are in tension, the equivalent point of application of T₁ and T₂ must be within the respective walls. Thus, the variables c₁ and c₂ are bounded c₁, c₂ ∈[0,1]. As will be seen in the following, the walls should be thin, and therefore the stress distribution can generally be considered to be relatively uniform, leading to values of c₁ and c₂ near ½. However, the specific point of application does not affect the subsequent derivation, and therefore need not be considered further.

The description of the cross section with d, x, and b is convenient, as d is generally a parameter determined by constraints from the environment, and then the design study involves selecting the variables x and b. It should be noted that the variables x, b and d are then geometrically bounded. In particular, x>0,b>0,d>x+b. Thus, some of the constraints are coupled. It should also be noted that for extending devices to operate Fₙ<px.

The device can be subjected to any combination of external forces and moments. The point of application of Fₙ is determined by the specific external forces in each scenario. The contribution of Fₙ to the moments equation in (1) depends on the distance between the line of application of Fₙ and the line of application of I₂. The F_{T}, applied may thus influence the M that can be supported, and vice versa. However, maintaining the contribution of Fₙ to the moments as a separate force with a certain point of application is desirable as it shows the moments and equivalent moments generated by Fₜ that can be supported by a design, and the effect of Fₙ on M.

Equilibrium Discussion

Equation (1) indicates that b affects the contribution of Fₙ to M through the term Fₙb(1−c₂), which has an effect on the device's performance. However, this is due to the fact that changes in b involve displacing the point of application of T₂. Equivalent alternatives for displacing the point of application of T₂ relative to the point of application of Fₙ include displacing the entire wall 6, or displacing the entire device. However, any possible offset of the external forces relative to the device to improve performance is considered to be already applied in practice. The problem of interest in terms of design is to maximize performance for a given external loading. In this regard, the effect of varying b on Fₙb(1−c₂) is not relevant from a design perspective as it is equivalent to offsetting the device, and it is therefore disregarded in the design derivation.

The cross section where equilibrium is considered is arbitrary, and therefore the analysis can be applied to any cross section on the device. This provides insight into the mechanical behavior of the entire device, and therefore it serves to study the design.

The equilibrium of forces also shows that external forces parallel to the cross section must be supported at the boundary where the device is isolated. Considering the definition of fluid, the direction of the pressure force is always normal to the boundary. Thus, the lateral forces must be supported by the structure in any design, particularly by Tₜ₁ and Tₜ₂ The contribution of this shear stress to the deflection, however, is considered to be relatively small, following the standard study of structures. In this regard, the equilibrium in the direction parallel to the cross section is not considered further.

The system of equations (1) provides the reactions T₁ and T₂ for any M and p given a design. These solutions, however, correspond to different structural deformations and therefore different displacements. Thus, the equilibrium alone cannot be used to determine the design to maximize M, as a combination of T₁ and T₂ to increase M always exists, but it may correspond to an undesirable deflection. In order to study the design for a given deflection of interest, a condition imposing a desired deflection to be maintained is required.

Deflection Condition

The purpose of the deflection condition is to define the relation between T₁ and T₂ that must be satisfied for a desired deflection to remain constant. In particular, the deflection must remain constant despite variations in the external forces and moments, as well as pressure applied.

Deflection depends on the differential wall extension. Thus, deflection can be maintained even at different pressures provided that both walls extend. The deflection condition can therefore not be determined from a specified extension value at each wall, but rather must be derived from a ratio between the extensions of both walls.

In order to attain a desired deflection, even without external forces or moments, a certain extension at each wall is necessary, which corresponds to the initial extension of the walls. Once the initial deflection is achieved, it can be maintained even for variable external forces and moments by compensating with pressure. More specifically, deflection can be maintained at variable values of wall extension provided that any increase in length in a wall is accompanied by a certain increase in length at the other wall. A condition to maintain a deflection can therefore be obtained by imposing the increase in length at both walls to be related through a certain ratio R as

δ₁=Rδ₂ (2)

where Sᵢ denotes the increase in length in wall i with respect to the length necessary to attain the initial deflection. The value of the ratio R is generally close to 1, but it can depend on the desired deflection. However, the derivation in this work does not require the exact value of R, and it is therefore not specified. It should be noted that any variation in extension must be associated with a variation both in external forces and moments, and in pressure.

The extension in a wall depends both on the stress applied and the wall stiffness. In addition, the initial extension required in each wall to reach the initial deflection involves a certain initial tension Tᵢₒ for i=1,2. In this regard, the deflection condition cannot simply impose a relation between T₁ and T₂, but it must include the stiffnesses of the walls as well as the initial tension of the walls. The increase in extension Si in a wall i can be related to the increase in tension in that wall Tᵢ−Tᵢₒ through a variable stiffness sᵢ as

Tᵢ−Tᵢ₀=sᵢδᵢ (3)

The value of sᵢ can be difficult to determine, and it is not necessarily constant. In general, sᵢ can depend on the material, the design, and the deformation. However, the specific sᵢ is not calculated here since it is not necessary for the derivation.

Substituting the relation between extension and tension (3) into (2), the condition that must be satisfied for a deflection to be maintained is obtained as

$${T}_{2} = \frac{{T}_{1} {s}_{2}}{{Rs}_{2}} + {T}_{20} - \frac{{T}_{10} {s}_{2}}{{Rs}_{1}} \qquad ( 4 )$$

The deflection condition is thus expressed as a relation between T₁ and T₂, as well as as set of parameters.

This condition (4) is applicable to any scenario with any desired deflection and combination of external forces and moments. The two terms on the right depend on the conditions to achieve initial deflection, and thus the desired deflection in each scenario is imposed by these terms. These two terms are constant. The value of R may also vary to some extent for some of these different scenarios, although in some instances the value of R can be equal for different deflections. Still, all these parameters are specified for a given scenario. Thus, (4) defines the relation between T₁ and T₂ that guarantees the deflection to be maintained in any scenario.

Interestingly, in the case of infinite stiffness at wall 4, the deflection condition (4) simply imposes T₂ to be constant. This is a typical situation as will be seen in the following, where designs with infinite wall 4 stiffness are particularly relevant. However, a constant T₂ is not a valid condition to maintain deflection in general, since, in extending devices, wall 6 may need to extend to a certain degree as pressure increases in order to compensate the extension in wall 4.

Design Derivation

The equilibrium and the deflection condition can be combined to analyze the design problem and derive a set of design principles, as described in the following.

Preliminary Qualitative Considerations

The equilibrium analysis, illustrated in FIG. 4a, indicates that the moment at the cross section necessary to support external moments as well as the equivalent moments generated by external forces is created between the pressure and the reactions. In particular, since pressure can only act in one direction, and the structure generally only acts in the opposite direction, the moment is created between the pressure pushing and the structure pulling.

The main challenge is supporting forces and moments that tend to reduce the deflection, i.e, forces and moments that contribute as positive values of M. Opposite forces and moments increase the deflection, and supporting them is thus trivial.

The pressure is always acting between the two walls in tension. Thus, the moment must be created between T₁ pulling and p pushing. T₂, on the other hand, opposes to this moment, and is therefore undesirable in general. The only purpose of wall 6 is to contain the pressurized fluid.

This qualitative analysis indicates that maximum T₁ and minimum T₂ are desirable. This could lead to the impression that concentrating the pressure application near wall 4, for example using thick or even hollow structure in wall 6, maximizes performance, as it maximizes T₁ and minimizes T₂. However, this arrangement also promotes an undesirable deflection. In the extreme case, a design with T₂=0 and thus T₁=px would be possible, but it would yield zero or negative deflection, which is undesirable as deflection must be maintained. Conversely, concentrating the pressure application close to wall 6 would minimize the increase in T₁ and thus the reduction in deflection when pressure is increased, enabling p to compensate generate the majority of the moment without loss of deflection. However, this also results in low Tᵢ and thus low forces and moments that can be supported. The analysis combining the equilibrium (1) and deflection condition (4) is derived in the following to resolve these design questions.

Detailed Analysis and Derivation

Imposing the condition requiring a deflection to be maintained (4) into the equilibrium of forces in (1) yields

$${T}_{1} = \frac{px - {F}_{n} - \text{?}}{1 = \frac{\text{?}}{\text{?}}} \\ \text{?} \text{indicates text missing or illegible when filed} \qquad ( 5 )$$

where κ=T₂₀−T₁₀S₂/RS₁ which corresponds to the initial deflection conditions. Substituting (5) into the equilibrium of moments in (1), the M that can be supported for a given design and a certain deflection is obtained as

$$\frac{px - {F}_{n} - \text{?}}{1 = \frac{\text{?}}{\text{?}}} [ {c}_{1} d + x ( 1 - {c}_{1} ) + b ( {c}_{2} - {c}_{1} ) ] - p \frac{{x}^{2}}{2} - {pxc}_{2} b + {F}_{n} ( h - b ( 1 - {c}_{2} ) ) = M \\ \text{?} \text{indicates text missing or illegible when filed} \qquad ( 6 )$$

It should be noted that, as previously mentioned, T₁>0 for operation to be possible since otherwise the structure would be in compression, and the device would not act as an extending device but rather as a passive structure. Thus, from (5), this implies a bound px−Fₙ−κ>0.

Expression (6) enables determining the design to maximize the desired performance, which in this case involves maximizing M. Expression (6) is applicable to any deflection, and therefore it can be used to address the design problem in any scenario.

The design principles can be extracted by considering the contribution of the design variables to M in (6). The stiffnesses s₁ and s₂ appear only as a ratio s₂/s₁. The ratio only contributes to the denominator of a term that should be maximized for the case of interest px−Fₙ−κ>0, and therefore s₂/s₁ should be minimized. As previously mentioned, the values of s₁ and s₂ may depend on the design as well as the material. However, in soft robotic devices, the material can generally be chosen to provide any desired stiffness, particularly including low values. In this regard, the material can be used to select s₁ and s₂, compensating for any variation in stiffness associated to the geometry. Thus, the minimization of s₂/s₁ is considered to be attainable with the material choice, independently of the rest of the design.

The variables s₁ and s₂ represent the overall stiffness of a wall, but the local stiffness within the wall needs not be constant. The specific stiffness distribution affects the line of application of T₁ and T₂, and therefore can be used to modify c₁ and c₂. The line of to application is determined by the location where the moment generated by the distributed stress within a wall is equal to that created by Tᵢ or T₂. For a given wall in extension, corresponding to a deflection, the normal stress within the wall can be considered to be strongly dependent on the local stiffness, especially if the stiffness distribution over the cross section presents significant differences. Thus, the wall layers with markedly higher stiffness generally involve higher local stress, and the line of application of the equivalent force can be considered to tend to these layers.

The stiffness distribution can therefore be used to modify c₁ and c₂. However, it should only be used for c₁. Considering that s₂ should be minimized, and that low stiffness is difficult to attain, any stiffness variation typically involves an increase in s₂, reducing the performance. Instead, a high s₁ can generally be maintained since local stiffness can typically be increased to compensate local reductions. Equation (6) indicates that a high c₁ is desirable, and therefore wall 4 should have a high stiffness in the outer layers and lower stiffness in the inner layers. Still, this is only relevant in designs where wall thickness is substantial, which are typically not the designs of interest, as shown in the following.

For a s₂ Rs₁ that is minimized, κ is typically negligible, as can be seen from the analysis in the next subsection. The derivation of the rest of design principles can then be divided in two cases for clarity of exposition.

Case with F_{T}=0 and Negligible κ

A case with Fₙ=0 and κ negligible can be considered first as it represents a common scenario of interest where the robotic manipulator must support external forces in the direction of bending, as in a nearly horizontal robot segment supporting and moving a payload against gravity, or a nearly horizontal segment moving a set of additional segments stacked serially at its distal end, which generate an external lateral force and moment. In addition, the case with Fₙ=0 and κ negligible provides a first intuitive understanding of the design principles. In this case, and for s₂/Rs₁ negligible relative to 1, each of the variables b,x,d only affects one or a small number of terms in (6), and thus can be easily determined. In addition, p can be factorized, so the desired value of these variables is independent of pressure.

The variable b affects three terms, the combination of which always reduces M since s₂/Rs₁>=0, and x, c₁ and c₂ are non-negative. Hence, b should be minimized, which can be written as b=0. Then, for b=0, the value of x to maximize M depends on c₁, s₂/Rs₁ and d. If 1−2c₁−s₂/Rs₁>0 then x should be maximized, and therefore x=d. If 1−2c₁−s₂/Rs₁<0 then the value of x to maximize M is

$$x = \frac{2 {c}_{1} d}{2 {c}_{1} + s 2 / Rs 1 - 1} \qquad ( 7 )$$

Considering that s₂/Rs₁ should be minimized, this implies x=d. Thus, in a design where s₂/Rs₁ is minimal, the most suitable cross section is x=d regardless of the value of the parameters c₁ and c2. The design of the cross section with maximal x and minimal b, so that the cross-sectional region corresponding to the pressurized fluid is maximal, in designs were s₂/Rs₁ is minimized, represents another relevant design principle. It should be noted that in some practical cases it may not be possible to minimize s₂/Rs₁ due to manufacturing or material constraints. In these cases, x is determined by (7), which may be lower than x=d.

Finally, the parameter d is determined by the practical application, but expression (6) highlights that increasing d results in higher force. Thus, d should be maximized to occupy all available room in each scenario.

Case with Fₙ₁κ≠0

Considering a general scenario including Fₙ and κ, a similar analysis can be applied to determine the design principles. In this case, the contribution of Fₙb(1−c₂) to M in (6) should be disregarded, as discussed in subsection 4.1. Then, for a s₂/Rs₁ negligible relative to 1, the variables x and b can be analyzed in conjunction. The analysis is divided in two further cases depending on the sign of Fₙ+κ.

For Fₙ+κ<0, the terms in (6) containing either of these variables can be aggregated into three groups: terms containing xb, terms containing sums of x and b and terms containing only x, as

$${τ}_{1} = - {pxbc}_{1} \\ {τ}_{2} = ( - {F}_{n} - κ ) [ {c}_{1} d + x ( 1 - {c}_{1} ) + b ( {c}_{2} - {c}_{1} ) ] \\ {τ}_{3} = px \frac{x ( 1 - 2 {c}_{1} ) + 2 {c}_{1} d}{2} \qquad ( 8 )$$

The terms corresponding to T₁ reduce M and should therefore be minimized, which entails that either x or b should be minimized. The term corresponding to τ₂ should be maximized which, considering that b+x<d, implies that combinations of x and b that yield b+x=d are desirable. In particular, if a trade-off between x and b is possible, combinations with higher values of x are preferable since the contribution of x to T₂ is higher. Finally, the terms corresponding to τ3 contribute to M, and should therefore be maximized.

The value of x to maximize τ₃ deserves consideration as the relation between τ3 and x is parabolic. If c₁>1/2, then τ₃ is maximized with the specific value of x

$${x}_{m} = - \frac{{c}_{1} d}{1 - 2 {c}_{1}} \qquad ( 9 )$$

which is always xₘ>=d. Considering the constraint x<d, the value of x should then be x=d. If c₁<1/2, then τ₃ as a function of x is a parabola that tends to infinity and intersects the x axis at 0 and at a negative value. Hence, x should also be maximized. Thus, for any c₁ within the possible values, if s₂/Rs₁ can be minimized as previously discussed, then the x to maximize τ₃ should be x=d.

The desirable values of x and b can thus be determined. τ₁ requires either x or b to be minimized, r₂ indicates that a trade-off between x and b be achieved, prioritizing x, and τ₃ requires x to be maximized. Hence, b should be minimized, which can be expressed asb=0, and x should be maximized, yielding x=d.

For Fₙ+κ>0, a similar derivation can be used. Defining a change of variable y=px−Fₙ−κ, the terms in (6) containing either y or b can be aggregated into three groups: terms containing yb, terms containing only b and terms containing only y, as

$${τ}_{1}^{′} = - {yc}_{1} b \\ {τ}_{2}^{′} = - ( {F}_{n} + κ ) {c}_{2} b \\ {τ}_{3}^{′} = {yc}_{1} d + \frac{{y}^{2}}{p} ( \frac{1 - 2 {c}_{1}}{2} ) - \frac{y ( {F}_{n} + κ ) \text{?}}{p} \\ \text{?} \text{indicates text missing or illegible when filed} \qquad ( 10 )$$

The terms corresponding to both—τ₁′ and τ₂′ reduce M, and thus should be minimized. Instead, the terms corresponding to τ₃′ increase and should be maximized.

As in the previous case, the maximization of τ₃′ requires some consideration. If c₁<1/2, the relation between y and τ₃′ is a positive parabola that intersects the y axis at 0 and at a negative value, since Fₙ+κ<px. Thus, y should be maximized. If c₁>1/2, τ₃′ as a function of y is a negative parabola that is maximized at

$${y}_{m} = - \frac{{pc}_{1} d - ( {F}_{n} + κ ) \text{?}}{1 - {c}_{1}} \\ \text{?} \text{indicates text missing or illegible when filed} \qquad ( 11 )$$

which is always yₘ>pd−Fₙ−κ. The value of y, however, is bounded 0<y<pd−Fₙ−κ since px>Fₕ+κ and x<d. Thus, for c₁>1/2, y should also be maximized.

The desirable values of y and b to maximize τ₃′ and minimize τ₁′, τ₂′ are then maximum y and minimum b. Reversing the change of variable y=px−Fₙ−κ, this implies b=0 and x=d.

The design principles for all admissible values of Fₙ+κ are therefore equal to those in the case where Fₙ=0. Hence, these constitute general principles to maximize the M that can be supported at a given deflection.

Final Derivation Considerations

This analysis was derived considering the equilibrium in an arbitrary cross section, and therefore it is applicable to any cross section on the device. In addition, it also applies to any deflection, pressure, and combination of external forces and moments. Thus, the design principles can generally be used to determine the design of a device to maximize the forces and moments that can be supported.

For a given design and deflection, both the reactions at the cross section and p vary with any external forces and moments applied to create the moment that maintains equilibrium. Specifically, for an increase in M, both p and T₁ must increase. If s₁ is not negligible, then the increase in T₁ is accompanied by an increase in T₂ that maintains deflection, with a ratio that depends on s₂/Rs₁. However, as discussed in the previous subsections, s₂/Rs₁ should always be minimized, and therefore the increase in T₂ is generally low.

The moment created by the external forces can vary in different cross sections. However, the design to maximize performance remains equal in all cross sections regardless of the equivalent moments, as argued in the previous paragraphs. The variable moment in different cross sections can result in uneven deformation along the device, but that simply implies a small variation in R, which does not affect the derivation. Thus, a constant cross-sectional design throughout the device, with a design determined by the design principles derived in previous paragraphs, is the most suitable design solution in general.

Derivation Discussion

In designs determined by embodiments of the invention, bending is mainly achieved with a differential stiffness in the two sides of a structure, rather than an asymmetric geometry. The values of T₁ and T₂ can therefore be equal, but the different longitudinal stiffness in both walls produces the deflection. In addition, when external forces and moments are supported, T₂ can be lower than T₁, but the deflection can be maintained thanks to the different stiffness in both walls.

The designs principles derived here are valid for T₁ and T₂ with a line of application anywhere within the wall thickness. Thus, even singular designs with a hollow wall structures to create separation are considered, but these are undesirable according to the design principles, which is a consequence of the fact that maximizing the cross-sectional region corresponding to the pressurized fluid is always desirable. In this regard, the results of the design analysis are general in terms of maximizing the force of the device at a given deflection.

The analysis indicates that the forces and moments that can be supported depend on the maximum pressure. Thus, if the pressure limit was infinite, the device would be capable of supporting practically any forces and moments, which illustrates the potential of soft robots with fluidic actuation. Still, elongation of the device would occur for finite S₁, complicating the practical implementation.

It should also be noted that the design principles only require the ratio s₂/s₁ to be minimized, but the absolute value is not imposed. This could lead to the false impression that the absolute stiffness is not relevant to the device performance. However, the absolute stiffness affects the pressure required to reach the desired deflection, as described in the following.

Initial Deflection

A similar approach as that described above is applied here in order to study the most suitable design to attain a desired deflection with minimum pressure. The same equilibrium of the device isolated at an arbitrary cross section can be considered, as illustrated in FIG. 4a. This provides the reactions for a given cross sectional design.

Deflection is achieved with a differential extension of the walls. This can be attained with either a difference between T₁ and T₂, a difference in stiffness of the walls, or a combination. The absolute extension in a wall i, denoted by Δᵢ, can be related to the tension using a similar expression as (3), but here in absolute terms

Tᵢ=sᵢΔᵢ (12)

As mentioned above, sᵢ can be difficult to determine, but the specific value is not necessary for the derivation, and is therefore not considered further. The use of (12) is advantageous as it elucidates the two methods to achieve deflection.

In order to attain the desired deflection with minimum pressure it is necessary to facilitate achieving the desired difference between Δ₁ and Δ₂. Using (12), the desired differential extension of the walls can be expressed as

Δ₂−Δ₁=T₂/s₂−T₁/s₁ (13)

Thus, in terms of stiffness, the difference between s₁ and s₂ should be maximized. It should be noted that maximizing the difference between s₁ and s₂ facilitates attaining the desired deflection regardless of the tensions in the walls. In this regard, it represents a general principle in terms of attaining the desired deflection at minimum pressure.

In terms of tensions, (13) elucidates that difference between T₁ and T₂ should also be maximized for a given p. Since sr should be maximized and s₂ minimized, the determining factor in (13) to maximize deflection is T₂, which should be maximized. Considering the equilibrium (1), and after some manipulation, it can be seen that the tensions depend on the cross section design as

$${T}_{1} = \frac{p \frac{{x}^{2}}{2} + {pxc}_{2} b + M + {F}_{n} ( h - {c}_{2} b )}{x ( 1 - {c}_{1} + {dc}_{1} + b ( {c}_{2} - {c}_{1} ) )} \\ {T}_{2} = \frac{p \frac{{x}^{2}}{2} ( 1 - {c}_{1} ) + {pxc}_{1} ( d - b ) - M + {F}_{n} ( h - {bc}_{2} )}{x ( 1 - {c}_{1} ) + {dc}_{1} + b ( {c}_{2} - {c}_{1} )} \qquad ( 14 )$$

The contribution of the term Fnb(1−c₁) is disregarded since it is equivalent to offsetting the device. Then, from (14), it can be seen that for M>0, which are the equivalent external moments of interest as previously discussed, reducing b to increase x is always desirable since δT₂/δb<0 and δT₂/δx>0. Thus, in order to maximize T₂ and therefore deflection, b should be minimized and x should be maximized, which can be written as b=0 and x=d. For x=d, (14) also elucidates that a maximum d is desirable, hence d should be selected to occupy all space available.

Interestingly, the performance in terms of initial deflection depends on the absolute stiffness of the walls, as elucidated in (13). Hence, for a given difference between s₁ and s₂ that cannot be increased, the absolute stiffness should be minimized to achieve deflection at minimum pressure.

This analysis therefore indicates that the design principles to attain a desired deflection with minimum pressure are maximum s₁, minimum s₂, b=0, x=a, maximum d, and minimum absolute stiffness when possible. It should be noted that these equalities in practice denote that the variables should tend to the desired values, i.e. minimum wall thickness and maximum region corresponding to the pressurized fluid. In the optimal design x=d, (14) indicates that T₂ increases with a, which should therefore be maximized to occupy all available room in each scenario. The derivation of these results is independent of the desired deflection or the pressure, and therefore they represent general principles.

Complete Design

The designs to maximize the external forces and moments that can be supported at a given deflection and maximum pressure, and to achieve a deflection at minimum pressure were elucidated in the two previous subsections. The design objective in this work involves attaining a desired deflection and maximizing the forces and moments that can be supported with a given maximum pressure, which couples both analyses.

Fortunately, there is an agreement in the design principles to achieve both objectives, as summarized in the following. The ratio sᵢ/s₂ should be maximized in both cases, which can be attained, for example, with a pleated structure in wall 4. Then, for a high s₁/s₂, x in both cases should be maximized, d should be maximized, and b should be minimized. The only difference is that the absolute values of s₁ and s₂ are not relevant in terms of maximizing force at a given deflection and maximum pressure, but they are relevant to attain the desired deflection at minimum pressure. Thus, absolute stiffness should generally be minimized.

This applies to any cross section on the device, and to any deflection and pressure value. Thus, these design principles summarized in the previous paragraph can be used to determine the most suitable design. Since the design principles are independent of the maximum pressure and the deflection, the most suitable design is relatively independent of the desired application.

Generalization to 3D

The study up to this point considered a planar scenario. The generalization to 3D is presented in this subsection. The analysis in 3D is mostly analogous; it involves considering the equilibrium of a device isolated in a cross section, aggregating the distributed reactions onto two tensioning force variables, distilling a condition to maintain deflection, and combining them in order to determine the design. However, the generalization of elements such as the aggregation of forces and deflection condition requires a careful examination.

In the 3D scenario, the soft robotic manipulator is considered to bend in a desired plane. External forces are considered to act in the plane of bending, as it represents the most relevant case for the design study. This scenario lends itself to the analysis of symmetric designs, but this symmetry is not used in the derivation in order to maintain generality of the study. The study can then be directly extrapolated to the design of devices capable of supporting out of plane forces.

The 3D device isolated in an arbitrary cross section can be considered, as in 2D. Here, the force associated to the pressure is pA, where A is the area of the cross section corresponding to the chamber, and p is pressure as before. The force pA is applied at the center of pressures, which depends on the chamber geometry.

Aggregation of forces T₁ and T₂

The distributed normal stresses at the cross section can also be aggregated into two forces T₁ and T₂ as in the planar case. However, the specific division of the cross section into two regions, the stresses of which correspond to T₁ and T₂, affects the analysis, and therefore must be considered. The moment at the cross section that produces bending and supports external moments and equivalent moments generated by external forces is created between the pressure and distributed reaction stresses at one side of the structure, with the reactions at the other side opposing to it. Thus, a suitable dividing line is that passing through the center of pressures and perpendicular to the bending plane, as it yields a T₁ aggregating all distributed stresses that contribute to the moment, and a T₂ aggregating all stresses that oppose to it, as in the planar scenario.

A dividing line passing through the center of pressures implies that the relative location of this line can vary with the cross-sectional design. However, this is desirable, as the cross-sectional stresses that contribute to the moment also depend on the design. Thus, the dividing line proposed here ensures that the stresses are appropriately aggregated, since the stresses associated to each force always share a common objective in terms of contribution to the device performance.

It should be noted that, as in the planar case, the equivalent line of application of T₁ and T₂ can be assumed to be within the region of the cross section they correspond to. Indeed, considering that extending devices achieve deflection thanks to a differential extension of the walls, and that this is produced with a pressurized fluid, it can generally be assumed that the normal stresses at the cross section are predominantly tensioning stresses, and therefore T₁ and T₂ are applied within the cross section.

Effect of Stiffness Distribution on T₁ and T₂

The specific line of application of T₁ or T₂ is affected by the stiffness distribution in the region they correspond to, as illustrated in FIG. 4b. As in the planar case, the stiffness in a region needs not be constant, and specific stiffness distributions can be used to displace T₁ and T₂. T₁ and T₂ are applied at the point where the moment they create is equivalent that generated by the normal stress in their corresponding region. In designs with a constant cross section and at a certain deflection, the local stress in the cross section can be considered to be higher at the sub-regions with markedly higher stiffness, particularly when the variations in the stiffness distribution are significant. Thus, the line of application of T₁ and T₂ can be considered to tend to the location of higher stiffness within their regions.

As in the planar case, the desired stiffness is considered to be selectable with the material choice, compensating for any effects from the design geometry. Thus, a typical configuration of interest with T₁ applied at an edge of the cross section can be attained with a high-stiffness material in the desired sub-region, and a lower-stiffness material over the rest of cross section, as shown in FIG. 4b. In this case, the line of application of Tᵢ can be considered to be relatively independent of the cross section geometry.

Generalization of Deflection Condition to 3D

The condition to maintain deflection can also be generalized to 3D. In order to maintain deflection, the overall normal strain distribution in the cross section should be approximately preserved, which implies that any increase in extension should be relatively homogeneous over the cross section. Considering that the stiffnesses at the cross section regions corresponding to T₁ and T₂ can be anticipated to be markedly different, a stress distribution with two distinct values corresponding to two regions in terms of stiffness can be expected.

The specific relation between T₁ and T₂ to maintain deflection is difficult to determine, as these average values may correspond to different stress and strain distributions. However, a ratio between T₁ and T₂ that guarantees that the deflection is maintained must always exist. Indeed, an increase in M while p and the external forces remain constant results in a decrease in deflection, whereas an increase in p while all external forces and moments are constant leads to an increase in deflection. Thus, a configuration where deflection is maintained exists, and this corresponds to a certain ratio between T₁ and T₂. In particular, following a similar structure as in 2D, at each configuration of equilibrium in each cross section, a relation of the type

$${T}_{2} = \frac{{T}_{1} {S}_{2}}{{RS}_{1}} + {T}_{20} - \frac{{T}_{10} {S}_{2}}{{RS}_{1}} \qquad ( 15 )$$

exists, which guarantees that the deflection is maintained with a certain value of R. It should be noted that the variables Sᵢ and S₂ denote the longitudinal stiffnesses of the cross section regions corresponding to T₁ and T₂, respectively, and are analogous to s₁ and s₂ in 2D.

The specific value of R can be difficult to determine, and may depend on the cross section. In general, considering the discussion in the previous paragraph, it can be bounded to be positive. Provided that it is positive, the specific value of R is not relevant to the design derivation in general, as in the planar case, and it is therefore not considered further.

It should be noted that that the existence of the condition (15) with a certain R is independent of the deformation distribution over the cross section. In some cross sections, it can occur that maintaining the deflection with different external forces and moments leads to a somewhat different strain distribution, resulting in a variation in the bending mode of the overall device. However, this only implies a somewhat different R in the cross sections, but the overall deflection is maintained. In addition, R remains positive in general, which is the main requisite for the derivation of the design principles.

Generalization of Design Derivation to 3D

With these concepts generalized to 3D, the equilibrium of the device isolated at an arbitrary cross section can also be considered in 3D. As in the planar case, the equilibrium indicates that T₁ and p generate the moment, and are desirable, whereas T₂ opposes to it. However, for a deflection to be maintained, relation (15) between T₁ and T₂ must be satisfied. The equilibrium of forces

T₁+T₂=∫∫_{A}p dA−Fₙ (16)

can therefore be combined with (15), and substituted into the equilibrium of moments, yielding

$$- ( {F}_{n} + K ) \frac{D}{1 + {S}_{2} / {RS}_{1}} + \int {\int }_{A}^{ } \frac{{D}_{p}}{1 + {S}_{2} / {RS}_{1}} - p χ dA + {F}_{n} H = M \qquad ( 17 )$$

where D is the distance between the line of application of T₁ and T₂, x is the distance in the direction of bending between a point in the cross section and the line of application of T₂, generalizing x in 2D, K is a constant associated to the initial deflection of the device, which generalizes K and is also typically low and positive, H is the distance between the line of application of Fₙ and T₂, generalizing h−b(1−c₂), and the rest of variables are a direct generalization of those in 2D. Both D and x depend on the design geometry and stiffness distribution. However, x does not depend on the line of application of T₁, and therefore is not affected by the stiffness in the region corresponding to T₁. The value of H can also vary with the design but, as in the planar case, this variation is disregarded since it is equivalent to offsetting the device. Expression (17) is equivalent to (6), and can be used to derive the design principles in 3D.

Case with Fₙ+K=0

Considering a case with Fₙ+K=0 first, expression (17) indicates that S₂/RS₁ should be minimized. Thus, maximal S₁ and minimal S₂ are desirable. As previously discussed, the overall stiffness in a region, S₁ or S₂, can be composed of different stiffnesses in different sub-regions, which can be used to displace the line of application of T₁ and T₂ towards the sub-regions of higher stiffness. Equation (17) indicates that D should be maximized while maintaining the values of x, i.e. by displacing the line of application of T₁ Hence, the stiffness distribution in the region corresponding to T₁ should be analogous to that in 2D, and consist of a high-stiffness sub-region near the edge in the direction of bending and a lower stiffness over the rest, as previously introduced and illustrated in FIG. 4 (left). This preserves a minimal S₂/RS₁, and maintains T₁ applied near the edge despite variations in the cross-sectional geometry.

The integrand in (17) can then be considered to be always positive. Its local value is the distance between T₁ and a differential element of chamber area, D−x, which is not affected by variations in the line of application of T₂. Hence, the integrand is relatively independent of design geometry, since the line of application of T₁ is relatively constant. Then, the area of the integral in (17) should be maximized in order to maximize M. This implies that the design should have minimum wall thickness, maximum chamber area, and a cross section that occupies all the available room.

As previously mentioned, a minimal S₂ is desirable. It should be noted that in very specific cases where the reduction of S₂ through material choice has reached the possible minimum, a cross section outline to some degree smaller than the room available may result in a noticeably lower S₂, and therefore improved performance despite the reduction in pA. However, these cases are generally unusual, and the performance improvement is typically low as the reduction in S₂/RS₁ is marginal. Hence, the design of a cross section to occupy all available room can be considered a general design principle.

Case with Fₙ, +K≠0

Considering a case with Fₙ+K≠0, a similar analysis can be applied. Here, for operation to be viable, Fₙ+K<pA. Thus, (17) indicates that S₂/RS₁ should be minimized. As in the case with Fₙ=0, (17) indicates that D should be maximized while maintaining the values of x, and therefore the same stiffness distribution in the region corresponding to T₁ applies. The point of application of T₂, however, is relevant in this case since an equal variation in D and x can modify M. If Fₙ+K>0, a high D is desirable despite an equal increase in x. However, a large A is also desirable, which can involve a reduction in D. Conversely, if Fₙ+K<0, a low D is desirable provided that x reduces equally. Still, an extensive A is also desirable with Fₙ<0 to maximize the contribution of the integral in (17), which can increase D and thereby reduce the performance. In this regard, the most suitable design depends on Fₙ, K, as well as the variation of D and x with A and the geometry. This design problem in the case Fₙ+K≠0 in 3D is analogous to that in 2D, but in 3D an ad hoc analysis is required to determine the 3D equivalents of c₁ and c₂ for a given geometry as well as K, and then generalize the design principles. This involves a numerical study that is beyond the scope of this work; thus, the specific geometry for each configuration under Fₙ+K≠0 remains as an open question.

Final Derivation Considerations

The derivation with both Fₙ=0 and Fₙ+K≠0 considered the equilibrium at an arbitrary cross section of the device. As in the planar case, the moment created by the external forces depends on the cross section, and therefore can vary along the device. However, the design study is equal despite variations in the external moments, and therefore applicable to all cross sections. Thus, the design principles can be applied to all cross sections, defining the most suitable design of the device.

It should be noted that the derivation of the design involves first establishing that the ratio between the stiffness of wall 6 and that of wall 4 needs to be minimized, and then determining the geometry. However, in the case that the ratio of stiffnesses could not be minimized, and for Fₙ=0, the design would then need to have an area of the cross section corresponding to the pressurized fluid not occupying all the cross section, which in 2D can be determined from (7). In 3D this can involve using structures to prevent cross section deformation, which can justify the introduction of braided chambers in some of the existing designs [19].

It should also be noted that, as previously discussed, the structure of extending devices is considered to extend only longitudinally, without expanding radially. The introduction of radial expansion would lead to contraction, which is undesirable in extending devices as it reduces the extension. Devices employing contraction are discussed in the following section. Extending devices should therefore maintain a constant cross section occupying all available space, which can be achieved by incorporating a set of braces or transversal fibers on the structure of the device.

Design of Contracting Devices

Design of Contracting Devices

The deflection in contracting devices is generated by a protruding wall, which forces one side of the device to contract, causing bending of the device. Thus, in contrast to extending devices, the pressure in contracting devices primarily serves to force a wall to protrude, and the moment for bending and supporting external forces and moments is mainly created between the tension in the protruding wall and the compression of another wall. The performance of the device depends on the design geometry and stiffness, which requires a detailed examination.

The design of contracting devices is studied using the same framework as in extending devices.

Equilibrium

Equilibrium Formulation

The equilibrium of a general contracting device isolated at an arbitrary cross section can be considered, as illustrated in FIG. 5. Imposing equilibrium of forces in the direction orthogonal to the cross section and equilibrium of moments with respect to the point where T₂ is applied, two equations are obtained

$${T}_{1} + {T}_{2} = px - {F}_{n} \\ {T}_{1} [ {c}_{1} d + x ( 1 - {c}_{1} ) + b ( {c}_{2} - {c}_{1} ) ] - \frac{{px}^{2}}{2} - {pxc}_{2} b - m 2 + {F}_{n} ( h - b ( 1 - {c}_{2} ) ) = M \qquad ( 18 )$$

where b, x, c₁, c₂, T₁, T₂, Tₜ₁, Tₜ₂, Fₙ, M and p are equivalent to those of extending devices. Equations (18) are analogous to those in extending devices, including the comments on the aggregation of external forces and moments into Fₙ, Fₜ and M, as well as the inequalities relating x, b and d based on geometric constraints.

In contracting devices, wall 6 must protrude and generate a contraction by puffing between its ends, whereas wall 6 must approximately maintain the initial length and bend. Wall 6 therefore serves as a backbone, which may undergo compression stresses. In particular, when T₁>px−Fₙ, wall 4 must be in compression, which typically occurs at low deflections. Hence, the structure of wall 4 typically needs to be capable of supporting compressive stress, and the stress distribution in wall 4 may combine tensioning and compressive stresses. The aggregation of these stresses is decoupled here into a moment associated to bending of the wall, defined as m₂, which can be generally considered to be negative and to reduce further with wall thickness, and the tensioning force T₂. This aggregation of the distributed stresses into m₂ and a normal force T₂ is generally admissible since, as will be seen in the subsequent presentation, wall 6 can generally be considered to act as a rod. The equivalent point of application of T₂ can thus be considered to lay within the wall thickness, 0<c₂<1, and typically near the center, c₂=1/2.

Equilibrium Discussion

As in extending devices, the equilibrium can be considered on any cross section of the device, and therefore the analysis derived from this equilibrium can be used to study the design of the entire device. Similarly, the equilibrium in the lateral direction also indicates that the structure of the device must support any lateral reactions in a passive manner. However, the effect of shear stresses on deflection is generally negligible, and and therefore not considered further.

The equilibrium equations (18) indicate that, in order to maximize the moment that can be supported, T₁ should be maximized and T₂ should be minimized, working in compression. Equations (18) also highlight that the pressure in contracting devices serves two separate purposes. First, and most importantly, it presses on wall 4 to create a protrusion, indirectly contributing to the equilibrium of moments through T₁. Second, it acts on the cross section, directly contributing to the equilibrium of moments as in extending devices. In this regard, contracting devices with equal diameter but different x can present different performance and the contribution px can be exploited. The direct contribution of px, however, also implies a higher tension at the walls, tending to reduce the protrusion, or equivalently limiting M, which couples both purposes of pressure.

The design in terms of geometry, including x and b, and stiffness, predominantly in terms of the protruding loran, must therefore be determined to maximize the M that can be supported.

Considering (18), configurations that attain high values of Min equilibrium with low or even zero p can be found. However, each of these equilibrium configurations may correspond to a different deflection. A condition imposing a desired deflection is therefore required in order to study the effect of design on performance, as in extending devices.

An important difference with respect to extending devices, however, is that in contracting devices the protruding wall is not perpendicular to the cross section along most of the device. The geometry of the protrusion therefore affects the device's performance, and must be first considered, as described in the next subsection.

Energy Considerations

General Energetic Analysis

The similarities between PAMs (Pneumatic Artificial Muscle) and contracting devices imply that some of the existing energetic approaches used in PAMs can be adapted for the study of contracting devices, and thereby extract insight into the behavior of contracting devices. In particular, energetic considerations can be used to elucidate the effect of some aspects of the design, such as structural stiffness, on the performance. Thus, specific aspects of the design, such as stiffness of the protruding wall, can be determined, defining specific protrusion geometries.

Energy conservation must be satisfied in a system corresponding to a general device with a given deflection and supporting general forces and moments. Virtual works can be considered for a structural deformation caused by a virtual element of fluid dV entering the device, with associated virtual increment of displacement dl at the point of application and in the direction of the resulting external force F, and associated virtual increment of rotation θ where M is applied. Considering an incompressible fluid, this yields

pdV=Fdl+Mdθ+dWₛ (19)

where dW, is the work required to deform the structure, which is dWₛ, >0. Equation (19) elucidates that any dWₛ reduces the forces and moments that can be supported, and therefore should be minimized.

In order to minimize dWₛ while maintaining operational capability, the longitudinal stiffness of the protruding wall should tend to infinity. The bending stiffness of the protruding wall, defined s_{b}, should either be s_{b}=0 over the entire protruding wall, or a combination of s_{b}=0 and s_{b}=∞ in different parts of the protruding wall. It should be noted that s_{b} must be s_{b}=0 at least at some parts of the protruding wall to enable operation. Finally, the bending stiffness of wall 6 should be minimal to minimize dWₛ, or equivalently to reduce the effect of m₂ in (18), but the wall should be capable of supporting compression stresses with minimal contraction.

The energy dedicated to deform the structure can be considered to be practically zero both in designs with only s_{b}=0 in wall 6 and in designs with a combination of s_{b}=0 and s_{b}=□ in wall 6, which renders both configurations equivalent in this regard. However, equation (19) also indicates that the forces and moments that can be supported with a given pressure are maximized when the dV that corresponds to a pair of dl, dθ is maximized. Thus, the geometry of the protrusion is relevant as it can increase dV for a given deflection. The protrusion geometry of designs with only s_{b}=0 in wall 4 is completely determined by the structural behavior. Instead, the protrusion geometry in designs combining s_{b}=0 and s_{b}=∞ depends on the distribution of s_{b}=0 and s_{b}=∞ and can therefore be selected. Considering that a maximum dV associated to a dl, dθ at the deflection of operation is desirable, the specific distribution of s_{b}=0 and s_{b}=∞ should be selected to maximize the dV associated to an increment of contraction of the protruding wall at the desired deflection, using all available room. This is generally determined geometrically considering that the wall geometry is composed of parts with predetermined geometry corresponding to s_{b}=∞, and parts with a specific geometry corresponding to s_{b}=0, which is a circumference arc as shown in the next subsection. The constraints from the environment and the desired deflection, however, depend on each scenario, and therefore the distribution of s_{b}=0 and s_{b}=∞ is specific for each application.

Braces and Braids

A set of braces can be used as an alternative design option to reduce the protrusion and adapt it to the environmental constraints. The braces need not involve any additional dWₛ provided that wall 4 is only comprised of parts with s_{b}=0 and s_{b}=cc and infinite longitudinal stiffness, although the braces should enable wall 4 to protrude to reach the desired deflection. The effect of these braces is thus analogous to that of a wall combining s_{b}=0 and s_{b}=∞, and therefore they represent an equivalent alternative to select the desired protrusion geometry.

Another design option for wall 4 in 3D scenarios is to include a braided structure, which may also minimize dWₛ. In particular, a braid that couples longitudinal and transversal tension (and therefore stiffness) through a certain ratio determined by the braid angle may also offer a performance equivalent to that of designs with infinite longitudinal stiffness provided that it requires minimal work to deform it. The braid then simply acts as a mechanism to transform transversal deformation into longitudinal deformation. This provides the capability of increasing contraction for a given protrusion, but it requires in-plane deformation in two directions, and thus a 3D structure. Considering that in-plane extension in the transversal direction is generally not desirable nor practical in soft robotic manipulators with contracting operation, and that braids generally involve a certain degree of dWₛ, the use of braids in wall 4 is considered disadvantageous over infinite longitudinal stiffness, and therefore not the main focus of this study.

Final Energy Discussion

The design in terms of stiffness can therefore be determined using energetic considerations, as described in previous paragraphs. The energetic considerations, however, do not directly imply a specific design in terms of x or b, since the relation between these and the maximization of dV, for a dl, dθ is difficult to determine a priori. The equilibrium approach introduced in the previous subsection can be used to determine the rest of design, and also to develop the study of contracting devices under the same framework as extending devices, but first a deflection condition is required.

Deflection Condition

An incompressible wall 4 is desirable in contracting devices, as argued in previous and following sections. Then, a deflection condition imposing the distance between the ends of wall 4 to remain constant suffices to ensure that deflection is maintained.

General Deflection Condition

The distance between the ends of wall 4 depends on the protrusion geometry and any extension of wall 4. As argued in the previous subsection, a maximal longitudinal stiffness is desirable for wall 4, and therefore wall 4 can be considered to be inextensible. In this case, the distance between the ends of wall 4 only depends on the protrusion geometry, which is generally a function of p, T₁, and s_{b}. For a given s_{b}, the distance between the ends of the protruding wall can thus be expressed as ζ, which a function of p and T₁.

The deflection is then determined by ζ. The specific ζ(T₁, p) can be difficult to determine in general as it involves solving a nonlinear structural problem with general boundary conditions. However, considering that ζ, and therefore deflection, depend on the protrusion geometry, insight into the structural behavior of the protrusion can be used in order to obtain a condition to impose a desired deflection.

In a general protruding wall, an increase in p for constant T₁ given s_{b} leads to a greater protrusion and more contraction, so dζ/dp<0. Conversely, an increase in T₁ for constant p and s_{b} tends to reduce the protrusion, hence dζ/dT₁>0. Thus, a relation between T₁, p and ζ generally exists as well for a given s_{b}, which can be defined as ƒ(p, ζ). Even though ƒ(p, ζ) is also difficult to determine in general, the function ƒ(p, ζ) can be either bounded or determined in specific designs of interest, which can suffice to obtain a deflection condition that enables a subsequent design study.

In particular, in designs with s_{b}=0, the equilibrium of a differential element of wall can be considered, as shown in FIG. 6, yielding

dm₁/dh=V₁

dV₁/dh=p−T₁ψ/dh

dT₁/dh=V₁dψ/dh (20)

where m₁ is the resulting moment at the cross section of the wall, V₁ is the resulting vertical force at the cross section of the wall, and ψ is an angle corresponding to the orientation of the cross section. For s_{b}=0, m₁=0. Thus, V₁=0, and therefore T₁ is constant over the wall region where s_{b}=0. Finally, the relation between T₁, p and the curvature radius of the wall, which can be defined as R=1/dψ/dh, is

T₁=pR (21)

The curvature of a wall or part of it over the region where s_{b}=0 is therefore constant. In this regard, the protrusion geometry in designs with purely s_{b}=0 is a circumference arc, whereas the protrusion geometry in designs combining s_{b}=0 and s_{b}=∞ is a combination of circumference arcs and the preselected geometry for the parts with s_{b}=∞. A bijective relation then exists between the geometry of wall 4 and the distance between its ends 4 in a given design, which is determined geometrically. In particular, in the case of a wall 4 with only s_{b}=0, a certain ζ implies a specific R. In the case of a wall 4 combining s_{b}=0 and s_{b}=∞, a given ζ also implies a certain R that is common in all regions where s_{b}=0, and is generally lower than the R in designs with only s_{b}=0 for an equal ζ.

The wall curvature is directly related to T₁ and p according to (21). Hence, a pair of T₁ and p imply a protrusion geometry, which in turn entails a certain ζ, and can therefore be used as a condition to impose a desired deflection. Equivalently, using the relation between R and ζ described in the previous paragraph for each particular design, (21) can be transformed into the condition

ƒ(p,ζ)=pR(ζ) (22)

where R(ζ) is determined geometricaily. Thus, for a given design in terms of distribution of s_{b}=0 and s_{b}=inf, the tension in wall 4 to maintain a desired deflection is proportional to p, and determined by (22) with the R corresponding to the regions where s_{b}=0. The condition applies to the entire wall. This includes regions where s_{b}=∞ since the moment at the ends of these regions is zero, and therefore the tension and its line of application within these regions is constant and equal to the tension at the ends.

The result that the wall geometry is specific for a certain distance between the ends of a protruding wall in wall designs with s_{b}=0 and infinite longitudinal stiffness is coherent with the energetic considerations. Indeed, if deformation energy cannot be stored in the structure, an increase in p and T₁ that maintains the distance between the ends of the wall and thus involves no motion of the device cannot result in any change in geometry in order to satisfy energy conservation.

Particular Designs with Braces or Braids

In designs including a set of braces, the deflection condition is similar. However, the specific design of the braces can lead to different values of tension in each segment of wall between two braces, particularly if the braces are not perpendicular to the cross section, resulting in different curvatures at each segment of the protrusion. The effect of the braces on the resulting wall tension must therefore be considered in order to then use condition (22) with the corresponding R. This effect can be determined by considering equilibrium at the point of attachment of the braces. However, it is not developed in this work since braces simply represent an alternative to modify the protrusion geometry equivalent to designs combining s_{b}=0 and s_{b}=∞, but do not provide specific performance advantages, as discussed in the previous subsection. It should be noted, however, that braces typically involve a reduction in R, which entails lower T₁ and therefore lower force for a given p and deflection, but also lower protrusion magnitude.

A deflection condition similar to (22) can also be obtained in designs with braids provided that s_{b}=0 is a valid assumption for the braid. However, condition (22) is derived considering a planar case, and a direct generalization to 3D only applies to protrusions with bending in a plane. Braids, on the other hand, couple transversal and longitudinal deformation, and therefore are intrinsically 3D.

Design Derivation

The equilibrium equations (18) and the deflection condition (22), together with the energetic considerations, can be combined to study the design, and derive design principles to maximize the forces and moments that can be supported.

Detailed Analysis and Derivation

First, the energetic considerations can be used to determine the wall stiffnesses of the design. In particular, the design should generally have an inextensible protruding wall with either s_{b}=0 or a combination of s_{b}=0 and s_{b}=∞ to maximize the dV associated to a dl, dθ at the desired operation deflection, using all available room. The specific combination of s_{b}=0 and s_{b}=∞ depends on each specific application, but typically larger regions of s_{b}=∞ provide higher dV and thus higher performance at low deflections, whereas larger wall regions with s_{b}=0 enable reaching and providing some support of external forces and moments at larger deflections. With these stiffnesses, the tension of the protruding wall (21) can be combined with the equilibrium of forces (18) in order to show that wall 4 must be designed to be capable of supporting compressive stress to allow operation at low deflections where R tends to infinity.

In these designs of interest, the equilibrium equations (18) can then be considered, and a desired deflection can be imposed by substituting (22), yielding

$$pR ( ζ ) cos α [ {c}_{1} d + x ( 1 - {c}_{1} ) + b ( {c}_{2} - {c}_{1} ) ] - p \frac{{x}^{2}}{2} - {pxc}_{2} b - {m}_{2} + {F}_{n} ( h - b ( 1 - {c}_{2} ) ) = M \qquad ( 23 )$$

where a is the angle between the direction of the resulting tensioning force in wall 6 and the direction normal to the plane of the cross section. (23) elucidates the fact that the design to maximize M depends on a, and therefore on the protrusion geometry. This is determined by the aforementioned stiffnesses, which maximize performance as discussed in subsection 5.2, and thus a is a specified value at each cross section.

Expression (23) provides the relation between the M that can be supported at a desired deflection and the design. (23) is analogous to (6) in extending devices, and can therefore be used to derive the additional design principles to meet the objective of maximizing M. Expression (23) is valid in general, and thus enables the determination of the design in a general scenario.

Case with Fₙ=0

A case with Fₙ=0 can be studied first, as it represents a common scenario of interest in practice, and is illustrative of the design principles. The effect of x, b and d on (23) is relatively decoupled in the majority of terms. However, their contributions to M depend on the values of c₁ and c₂, especially in terms of sgn(c₂−c₁), and therefore these two parameters must be first considered.

Specific values of c₁ and c₂ can be difficult to select with the design, but general tendencies for the desired values of the parameters can be considered, which can suffice for the design study. The value of c₁ affects M through three terms: a positive and two negative ones. However, considering that the variables x, b and d are related through x+b<d, it can be seen that the total contribution of c₁ to M is always positive, and therefore c₁ should be maximized to the extent it is possible with the design. The contribution of c₂ to M is more complex to analyze, and therefore its desired tendency is difficult to determine. However, considering that c₁ and c₂ are equivalent design parameters, their maximum values can be considered similar. Thus, in a design where c₁ is maximized, it can be assumed that c₂<=c₁.

The sgn(c₂−c₁) can then be considered to be negative. This implies that b tends to reduce M in (23). In addition, b also tends to reduce m₂, leading to more negative values, which reduces further M. Thus, b should generally be minimized, which can be expressed asb=0.

The contribution of x to M in (23) is then only through two terms, with a quadratic relation. Thus, the value of x to maximize M can be directly determined as x=(1−c₁)R(ζ). Considering the constraint x<d, the value of x should tend to d at low deflections where R→∞, even for a c₁ that is maximized, which can be expressed as x=d. At larger deflections, the required value of x may be lower than d. A design where x reduces with deflection can be considered to be inviable in practice unless pressures below atmospheric pressure are used, which is typically impractical as it limits the maximum pressure difference. Thus, at larger deflections, the design in terms of x should be selected for a specific operation according to x=R(ζ)((1−c₁).

Finally, the value of d is determined by the environment in each application. (23) shows that a high d is desirable, and therefore it should be the selected so that the device reaches the constraints from the environment at the maximum protrusion. This agrees with the aforementioned design of wall 4 to use all available room, although the specific wall geometry, determined by the regions with s_{b}=0 and s_{b}=∞, should be selected to maximize the dV associated to dl, dθ, as previously described.

Case with Fₙ≠0 The design in the general case Fₙ≠0 can be studied in a similar manner. In contracting devices, the deflection condition (22) only imposes a constraint on T₁, but does not involve T₂. As a consequence, the contribution of Fₙ to M in (23) is through a constant term Fₙh and a term depending on the design Fₙh(1−c₂). As in extending devices, the contribution of the term Fₙb(1−c₂) to M can be disregarded since it is equivalent to offsetting the device with respect to the external forces. The contribution Fₙh is fixed and equivalent to an additional external moment to be supported.

The design derivation in the case Fₙ≠0 is therefore analogous to that in the case Fₙ=0, and the principles for contracting devices both with and without external forces and moments are equivalent. These, together with the aforementioned principles corresponding to the stiffnesses of the walls, constitute the design principles to maximize the M that can be supported at a given deflection with contracting devices.

Final Derivation Considerations

It should be noted that, in designs combining s_{b}=0 and s_{b}=∞, the derivation also applies to the regions where Sₜ=∞. However, in these regions, both the line of application of T₁ and R(ζ) correspond to those at the boundaries with the adjacent regions, at the side where s_{b}=0. Thus, the line of application of T₁ needs not necessarily be within the wall in designs with curved, rigid wall regions. The design principles, however, indicate that d should be maximized within the room available, and x should generally occupy the entire cross section. Hence, the rigid parts in wall 4 should be straight, and the same principles derived in previous paragraphs apply.

Interestingly, the geometric principles indicate that the thickness of wall 4 and 6 should be minimized in the majority of cases. This is coherent with the principles in terms of stiffness indicating that the bending stiffness of wall 4 should be minimal while supporting compression stress, and the bending stiffness of wall 4 should be minimal in the desired regions. Thus, the resulting designs can be produced in practice.

Derivation Discussion

This derivation confirms that wall 6 in standard contracting devices must undergo compressive stress when R(ζ)>=x−Fₙ/p, which typically occurs at low deflections. High values of x can aid in reducing the compressive stress but, in general, designs without the capability of supporting compressive stress in wall 6 cannot operate at low deflections. This is due to the fact that a protrusion generally involves a T₁.

The need for a wall 6 capable of supporting compressive stress can only be prevented by reducing the T₁ associated to a protrusion and p, which requires exceptional solutions. One of such solutions is to include an elastic sheet that acts as a continuous set of elastic braces opposing to the protrusion, thereby reducing T₁ and thus leading to a contracting device without the need for a wall 6 capable of supporting compressive stress. Such a design solution is relevant in the application described in section 6. However, in general such a solution also involves a reduction in the forces and moments that can be supported.

Initial Deflection

The design principles to attain a desired initial deflection with minimum pressure can be determined by following a similar derivation as that to derive the principles to maximize the forces and moments that can be supported.

First, the energetic considerations indicate that the structure should store minimum energy. This implies an inextensible protruding wall with either s_{b}=0 or a combination of s_{b}=0, s_{b}=Wall 6 should then be incompressible, and with minimum bending stiffness.

The study of the protrusion then indicates that the protrusion geometry is directly related to the deflection. Thus, the desired initial deflection can be imposed by selecting a protrusion geometry with a desired R, and using the deflection condition (22), where the specific R(ζ) is determined geometrically.

Equilibrium of moments can also be considered in a device at the desired initial deflection and with no external forces or moments. This is equivalent to the equilibrium in (18)b, shown in FIG. 5, particularized to Fₙ=0, M=0. The imposition of the desired initial deflection (22) to (18)b yields

$${pR}_{i} ( {c}_{1} d + x ( 1 - {c}_{1} ) + b ( {c}_{2} - {c}_{1} ) ) - \frac{{px}^{2}}{2} - {pxc}_{2} b - {m}_{2} = 0 \qquad ( 24 )$$

This equation can be used to determine the design to attain the desired initial deflection with minimum pressure. First, (24) indicates that, in order to minimize p, m₂ should be minimized, which agrees with the energy considerations. Then, factorizing p, it can be seen that the design to minimize p in (24) is equivalent to the design to maximize M in (23). Hence, the design geometry and stiffness should be equal to those derived in the previous subsection.

Complete Design

The design principles to attain a desired deflection at minimum pressure are equal to those to maximize the forces and moments that can be supported at a desired deflection, both with and without Fₙ. Thus, these represent the general design principles for contracting devices, and are summarized in the following.

The protruding wall should have infinite longitudinal stiffness, and either s_{b}=0 or a combination of s_{b}=0 and s_{b}=∞ to maximize the dV associated to an increment in the contracting wall, using all available space. Wall 6 should be incompressible with minimum bending stiffness. The parameter c₁ should be maximized to the extent possible. The total width d should be selected so that wall 4 reaches the constraints from the environment at maximum protrusion. And finally, the design geometry should be b=0, and x=R(ζ)(1−c₁), which is typically x=d.

Generalization to 3D

The design derivation presented up to this point can be generalized to 3D. The study in 3D is mostly equivalent: it involves using energy considerations to outline the device's stiffness, and then combining it with an equilibrium analysis to derive the design principles. However, some aspects of the generalization require a detailed analysis.

Generalization of Design Derivation to 3D

The energy considerations can be applied to 3D, showing that the structure of a 3D device should store minimum energy to maximize the forces and moments that can be supported. Thus, the structure of a 3D contracting device must be composed of two regions: a first region corresponding to a protruding wall, which should be inextensible and with either s_{b}=0 or a combination of s_{b}=0 and s_{b}=∞, and a second region of the device acting as a backbone, which should be incompressible and with minimum bending stiffness, equivalent to wall 6 in the planar case.

The cross section of the device must then be divided, with parts corresponding to these two structural regions. Unlike in extending devices where the role of the cross-sectional stress in the cross section is dictated by the position relative to the center of pressures, in contracting devices the purpose of the local stress in each element of area over the cross section is not clear a priori.

A cross section divided along an arbitrary curve can be considered. This defines the two regions in terms of stiffness, where one region corresponds to the protruding, inextensible wall, and the other region corresponds to the incompressible wall. The equilibrium of the 3D device isolated in this general cross section divided along an arbitrary curve can then be considered in an analogous manner as in subsection 5.1, with T₁ corresponding to the aggregated normal stresses in the region of the protruding wall, and T₂ corresponding to the aggregated stresses in the other region. The equilibrium indicates that, in order to maximize the forces and moments that can be supported, the separation between T₁ and T₂ should be maximized. Thus, the curve dividing the cross section must be selected to maximize the distance between T₁ and T₂ in the direction perpendicular to these forces and in the plane of bending. This specifies the purpose of each region of the cross section, and defines the stiffnesses of the device.

Equilibrium of the 3D device isolated in an arbitrary cross section with T₁ and T₂ defined by this dividing curve can be used to determine the rest of the design in an equivalent manner as in the planar case. The design involves minimizing the thickness of the region corresponding to T₂, maximizing the area of the cross section corresponding to the pressurized chamber for typical operation deflections, and maximizing the increment of volume in the device for an increment in contraction of the protruding wall at the operation deflection, using all available space.

The specific division of the cross section along a curve, or equivalently the allocation of the different parts of the cross section to the different regions, in order to maximize the distance between T₁ and T₂ depends on each scenario. In typical scenarios where the spatial constraints in a cross section are defined by a rectangle, wall 4 should correspond to one side of the rectangle, and wall 6 to the opposite side, as shown in FIG. 7 (a). In more general scenarios with any spatial constraints, wall 4 should correspond to the entire frontal region of the device when observed from the direction in which it bends, as illustrated in the example in FIG. 7 (b), creating a frontal protrusion, while wall 6 should correspond to the opposite side.

These designs oppose to designs with a wall 4 that extends to the lateral regions, such as that shown in FIG. 7 (c). Protrusion in the lateral direction, or in any direction different from a frontal protrusion, is generally undesirable. This can be elucidated using the equilibrium, as it generally involves increasing the region corresponding to T₁ to the laterals, which modifies the line of application of T₁, reducing the distance between T₁ and T₂. The undesirable lateral protrusions can also be explained using energetic considerations. The forces and moments that can be supported depend on the volume increase of the device (19) for a contraction increment. However, the geometry of the protrusion generally cannot be selected to adapt exactly to the volume available from the spatial constraints, which are commonly prismatic, leaving some volume unused. In designs with lateral protrusions, the unexploited volume is typically larger than in designs with only frontal protrusion, as unused volume appears at both sides or near vertices of the available room, leading to lower performance. Thus, both equilibrium and energetic considerations confirm that the protrusion should generally be only frontal.

Designs in 3D such as those in FIGS. 7 (a), (b) typically include lateral walls. However, these should not contribute to the protruding wall nor to the opposite wall in order to maintain the distance between T₁ and T₂ to a maximum. These lateral walls only serve to contain the pressurized fluid and enable protrusion of wall 4, but should not affect the structural behavior of the device. Thus, these walls should generally be designed to minimize any resistance to deformation while containing the fluid without protruding laterally, e.g. using a pleated structure with tendons connecting both laterals. In specific cases, however, these lateral walls can be used to reduce the T₁ associated to a deflection and pressure, reducing the compression on wall 6. This is equivalent to the use of an elastic sheet introduced above for planar designs, and is a relevant solution in the design presented in the following section.

Discussion of 3D Derivation

The design of contracting devices in 3D presented in this subsection elucidates that contracting devices are similar to a segment of continuum robot actuated by PAMs and with an elastic backbone. However, contracting devices integrate the different parts, and can be designed with the principles elucidated in this work to improve performance. Still, both contracting devices and devices including PAMs present the disadvantage of involving a protruding wall, which typically protrudes outwards, requiring additional room to operate.

Summary

The main design principles derived in the previous sections are summarized in the following, first for extending devices, and then for contracting devices. The overall procedure to design a soft robotic manipulator using the design principles is then outlined. It should be noted that this section is intended as a summary of the main principles, and the reader is referred to the previous sections for details and clarifications on the principles and their derivation.

Extending Devices

The design principles for extending devices in both 2D and 3D can be summarized as follows. The longitudinal stiffness in the region corresponding to wall 4 should be maximized, which can be expressed as maximal s₁ in 2D and equivalently maximal S₁ in 3D. The stiffness distribution should be selected so that the maximum stiffness is concentrated near the edge of the cross section in the direction of bending in order to displace the line of application of T₁ towards the cross section contour. The stiffness in the region corresponding to wall 6 should be minimized, which can be expressed as minimal s₂ in 2D, and minimal s₂ in 3D. This minimal stiffness can be achieved, for example, with a pleated structure. The total cross section of the device should be maximized to occupy all available room. The thickness of the walls should be minimized. Finally, the chamber area should be maximized, in general case were S₂/RS₁ is minimized, to ensure that the region of the cross section corresponding to the pressurized fluid is maximal. It should be noted that these last three principles apply to any 2D case and to the 3D case Fₙ+K=0. However, in the 3D case Fₙ+K≠0, the specific geometry of the cross section must be determined using numerical methods.

The performance of extending devices is related to their operation. In extending devices, the combination of T₁ and the direct contribution of pressure in the cross section create the moment that supports external moments and equivalent moments generated by external forces. Thus, the performance of extending devices tends to be relatively low at low pressures, but remains relatively constant as deflection and pressure increase. As a result, extending devices are relatively well suited to operate at large deflections and corresponding higher pressures.

Contracting Devices

The design principles for contracting devices in both 2D and 3D can be summarized as follows. The longitudinal stiffness of the protruding wall should be maximal. Its bending stiffness should generally be a combination of parts with infinite and minimal bending stiffness, selected to maximize the dV corresponding to an increase in wall contraction at the operation deflection, although in specific cases braids or braces can be used to maximize the dV associated to a contraction increase. Wall 6 should be capable of bending with minimum resistance while generally being capable of supporting compression forces. The distance between T₁ and T₂ should be maximized by selecting appropriate regions for walls 1 and 2, as illustrated in FIGS. 7a to 7c. This implies that in some cases lateral walls may be included, typically in the form of pleated structures with braces to prevent lateral expansion. However, these lateral walls should only serve to contain the pressure and not affect the structural behavior of the device. The total cross section should be maximized so that the device occupies all available room at the operation deflection, where the protrusion should be maximal. The thickness of the walls should be minimized. Finally, the region of the cross section with pressurized fluid should generally be selected to be maximal at the operation deflection.

The performance of contracting devices is also related to their operation. The support of external moments and equivalent moments generated by external forces is primarily achieved between wall 4, which is in tension thanks to the pressure forcing wall 4 to protrude, and wall 6 in compression. The direct contribution of pressure to the moment at the cross section is then secondary. As a result, their performance is relatively high at low deflections, where low pressures produce significant T₁, but tends to reduce at higher deflections, where the T₁ created by a given pressure is lower.

Outline of Design Principles in Methods According to Embodiments of the Invention

The design principles described above may be applied to methods according to embodiments of the invention in order to determine the most suitable designs for any given scenario.

Outline of Design Principles Application

Embodiments of the invention will now be described with particular reference to FIGS. 8, 9a, 9b, 9c and 9d, and 10.

By means of the present invention it is possible to determine the most suitable design for a given scenario. The most appropriate design for a manipulator will depend on multiple factors in terms of requirements and constraints of the scenario, so each case needs to be considered individually. Nonetheless, embodiments of the invention of the invention enable an overall design procedure to be used.

This overall design procedure according to embodiments of the invention is set out in FIG. 8 and described in more detail below.

Step i) of the method is designated generally by the reference numeral 80. In this step the conditions under which the manipulator will be operated or identified. Specifically, the spatial constraints and the scenario requirements (typically desired deflection) are considered.

Step ii) is designated generally by the reference numeral 82, in this step, the category of device is selected accordingly. If the desired deflection is relatively low and some space is available for a protrusion, a contracting device is selected. Conversely, if the desired deflection is high, or the maximum diameter is very constricted, an extending device is selected. If the desired deflection presents a broad range of values of interest, a device combining extending and contracting actuation can be selected. Finally, if the desired deflection is intermediate, both an extending and a contracting device need to be explored, and the most suitable design needs to be selected by comparing the performance of the final designs of both types of device.

Once the type of device is chosen, step iii) designated generally by the reference numeral 84 involves selecting the total cross section to occupy all room available at the desired deflection. In some cases, braids or braces may be introduced to adapt to the total cross section to the spatial constraints. Step iv) designated by the reference numeral 86 then involves designing a preliminary cross-sectional geometry, following the design principles and defining a preliminary estimate of the regions corresponding to each wall.

Next, step v) designated by the reference numeral 88 involves selecting the stiffness distribution following the design principles. In most contracting devices, this can affect the design of the total cross section to use all available room and any braids or braces associated with it, and thus they need to be designed in conjunction. Once the stiffness distribution and total cross section are established, the cross-sectional geometry is adjusted according to the design principlesin accordance with step vi) designated generally by the reference numeral 90. Iteration can then be conducted to satisfy all design principles to the best possible extent in accordance with the invention, and as shown in FIG. 8.

Thus, the method according to embodiments of the invention provides the most suitable design layout. In some cases, as determined through step vii) the design principles can show that a compromise is necessary, as not all principles can be concurrently satisfied. In addition, the value of specific design parameters may need to be optimized in accordance with step viii) designated generally by the reference numeral 94, which is also generally identified by the design principles. FE simulations can be used to optimize the parameters, and resolve the compromises, yielding the final design. The FE simulations can also be used to compare final performance of designs in the case that both an extending and a contracting device are explored, and thus select the best.

Application to Manipulate Design

A method according to embodiments of the invention as described hereinabove will be used in this section to derive a design of a soft robotic manipulator in a prototypical scenario with reference to FIGS. 9a, 9b, 9c and 9d, and 10.

Scenario Definition

A minimally invasive surgery (MIS) scenario requiring a soft robotic manipulator is selected as the prototypical scenario in this work. Soft robotic manipulators are well suited to MIS, offering compliance, modularity, compatibility with magnetic resonance imaging, and miniaturization possibilities that are particularly desirable in keyhole surgery. The recent interest in the subject illustrates the relevance of these devices in medical applications.

The specific requirements for the soft robotic manipulator in the selected scenario are for it to be able to bend laterally in any direction, providing 2 degrees of freedom (DOFs), and to maximize the lateral force that can be supported at deflections near 20 degrees. This deflection is measured as the angle between the centers of the manipulator's ends in undeformed and deformed configurations, and is selected arbitrarily to illustrate the determination of the design in a representative case. The outer diameter of the device is constrained to 6 mm, and the operation pressure is limited to 6 psi. For safety reasons these are typical values in MIS where a small diameter is required for entry into the body, and the maximum pressure is limited due to the relatively weak sealing at miniature size and to prevent damage in case of bursting. These values are also similar to the pressures and deflections considered in the literature for devices with similar characteristics.

The minimum wall thickness is considered to be limited by manufacturing constraints and associated resilience to puncture, leakage, and withstanding the maximum pressure. The manufacturing of soft robots commonly involves casting the hyperelastic structure of the device, adding fibers, sheets or other inextensible elements, and finally affixing all the elements typically with additional layers of hyperelastic material. Considering the typical tolerances associated to these processes, a minimum wall thickness of 400 μm is selected for the prototypical scenario. The suitability of this thickness to withstand the maximum pressure with a safety margin to cope with manufacturing tolerances while providing a certain degree of resilience is confirmed in the simulations above.

Design Derivation

Primary Design Derivation

The principles of operation of extending and contracting devices are different, which makes the devices suitable for operation at different deflections. Contracting devices predominantly support external forces and moments thanks to the pressure forcing the protruding wall to be in tension and thus the opposite wall in compression, and the direct contribution of pressure to support external moments is secondary. As a consequence, they generally offer higher performance at lower deflections where even low pressures create significant tension in the protruding wall. However, as deflection increases, the relation between T₁ and p reduces, and T₂ becomes a tension force, leading to lower performance. Conversely, extending devices support external forces and moments thanks to the direct contribution of pressure to generate a moment when considering equilibrium of a device isolated in a general cross section, in combination with T₁. Thus, they typically offer lower performance at low deflections and low pressure, but their performance remains relatively constant as deflection and pressure increase, offering higher performance at higher deflections. A design combining extending and contracting operation would therefore be advantageous in this application that requires operation at various deflections.

The design principles for extending and contracting devices share many similarities. The wall thickness should generally be minimized, and the area of the cross section corresponding to the pressurized fluid should be maximized; the devices should use all available room; the region corresponding to wall 4 should present a maximal longitudinal stiffness, and this should be concentrated near the edge to maximize the distance between the line of application of T₁ and T₂. In addition, these principles are generally independent of the desired deflection and pressure. Thus, a design combining both types of operation can be conceived for this scenario.

The main design difference is that, in extending devices, a wall 6 with minimum longitudinal stiffness is desirable, which can be attained with a pleated structure. Instead, in contracting devices, wall 6 must typically support compressive stresses and therefore a pleated structure is not viable. Thus, a certain degree of compromise is necessary.

In this prototypical scenario, any protrusion over 6 mm diameter is undesirable. Thus, the outer structure should be cylindrical with 6 mm diameter. In order to provide bending in any direction, the design must be 3D, and should then include at least three chambers in the cross section along the device. Since chambers involve partition walls that increase bending stiffness, the number of chambers should be minimized, leading to three chambers being selected. The design principles indicate that cross section deformation is desirable from an extending device perspective in order to maximize the area of the cross section corresponding to the pressurized chambers, and displace the line of application of T₁ towards the outer contour, with maximum concentration of stiffness at the region corresponding to T₁. Such cross section deformation leads to a protruding central rod. This can be exploited as the protruding wall in contracting devices. Thus, the central rod should have an infinite longitudinal stiffness, which is desirable for it to act as the protruding wall of extending devices, and as wall 4 of extending devices. This results in a device combining extending and contracting operation, with a design that is desirable for both types of operation as it maximizes area of the cross section corresponding to the pressurized fluid, and presents a desirable stiffness at the equivalent of wall 4.

Since the design includes contracting operation, a structure capable of supporting compressive stress is necessary to act as wall 6. The device must be capable of bending in any direction, hence the line of application of the equivalent of T₂ must be near the center of the device. The most suitable solution is then the incorporation of an outer cylindrical structure made of superelastic material such as nitinol, with notches in alternating perpendicular directions to enable bending with minimum resistance while supporting compression forces.

The most suitable design of the soft robotic manipulator is therefore a cylinder with a constant cross section that consists of three equal chambers that can deform and present a maximum area, and an outer metallic structure, as conceptually illustrated in FIG. 9a,

FIG. 9a illustrates a soft robotic manipulator designated generally by the reference numeral 900. As mentioned above, the manipulator 900 is substantially cylindrical in a non-deformed state. The manipulator 900 comprises three chambers 902 which, in this embodiment are all of substantially the same shape, size and volume.

The manipulator comprises an outer wall 904 which has an outer metallic structure. The manipulator 900 further comprises a rod 906 extending substantially axially through the manipulator 900. The ratio S₁/S₂ should be maximized according to the design principles, which implies a minimal stiffness at the outer wall 904, and maximal longitudinal stiffness at the rod 900. This can be obtained by designing an outer wall 904 made of minimal stiffness material and with minimum thickness, which in this scenario corresponds to 400 μm as described in the previous subsection, and including an inextensible thread at the rod 906. The chambers 902 are separated by partition walls 908. The stiffness of the partition walls should be minimal to facilitate cross section deformation, and wall of the partition thickness should be minimal to maximize the area of the chambers 902 in the cross section. Since the maximum cross-sectional deformation is limited by the outer wall 904, the partition walls 908 are always below the maximum strain of typical hyperelastic materials, and thus the minimum wall thickness in this scenario, 400 μm, can be selected. It should be noted that the outer structure serves to prevent radial expansion of the outer wall. The maximum protrusion of the rod 906 is also limited by the outer diameter, and therefore the device respects the diameter constraints while offering contracting operation,

Discussion of Primary Design Found

The design layout of this primary design obtained resembles that of the FMA (flexible micro-actuator), but the principles of operation and the specific geometry and stiffnesses are different. This layout combines extending and contracting operation, in contrast to the FMA that only involves extending operation. In addition, the wall thickness in this layout is lower than in the FMA to maximize the chamber area in the cross section, the central rod is inextensible to maximize force, and the design includes an outer structure to support compression forces. Finally, the partition walls in the proposed layout contrast with those in the FMA, as they are designed to facilitate cross section deformation, which maximizes the area corresponding to the pressurized fluid in the cross section and leads to contracting operation.

The manufacturing of the proposed outer structure capable of supporting compression forces is challenging, particularly at the miniature size of this prototypical scenario. In addition, it can introduce bending resistance, limiting performance. Furthermore, the structure can limit extending-type operation at relatively high pressures.

Alternative Design

An alternative design is illustrated in FIGS. 9c and 9d which show another manipulator 910 comprising a rod 912 extending axially along the axis of the manipulator and an outer wall 914. This design is easier to manufacture. The need for a structure to support compressive forces stems from the significant tension at the rod 902 associated with a protrusion, and mainly occurs at low deflections. This tension can be reduced by introducing some resistance to the protrusion. In this design, the resistance can be introduced by using partition walls 918 with some stiffness. Thus, by combining partition wall stiffnesses together with some extension of the rod 902, a design without compression force on the outer structure can be achieved. It should be noted that, in such design, the rod 900 still serves to increase performance by introducing contracting actuation and by maximizing S₁/S₂, hence a high central rod stiffness in the longitudinal direction is desirable. The main purpose of the partition walls 918 is to compensate any excessive effect of the protrusion for a given pressure and deflection, and therefore the partition wall stiffness (PWS) depends on the longitudinal central rod stiffness (LCRS), with higher LCRS requiring higher PWS.

The alternative design is therefore similar to the previous design, as conceptually illustrated in FIGS. 9a and 9b, but with different values of PWS and LCRS. This leads to a design without compression at the outer wall, which eliminates the need for complex structures while enabling operation at low deflection. Consequently, it represents the design selected for this prototypical scenario.

The outer wall 914 can then be made of soft material, and should have a minimal wall thickness to maximize the area of the cross section corresponding to the pressurized fluid, and a minimal bending stiffness to maximize S₁/S₂. In this embodiment, the outer wall 914 has a pleated structure with circumferential fibers in order to minimize bending stiffness. Alternatively, a cylindrical outer wall made of soft material with circumferential fibers to prevent radial expansion while allowing longitudinal deformation is practically equivalent and easier to manufacture, hence here is the solution selected. The material of the outer wall 914 should be hyperelastic, with low stiffness, and capable of withstanding pressure when combined with fibers. In order to consider a realistic material that is readily available, DragonSkin 10 (Smooth-On, USA) is selected for this prototypical scenario. This is a common material in soft robotics and it has been previously characterized in the literature. The wall thickness should be the minimum possible, which corresponds to 400 μm in this scenario, as described in the previous subsection. This wall thickness can withstand pₘₐₓ with only minor bulging of the rubber between the fibers, which corresponds to a maximum strain in the rubber below the failure limit of the material, as confirmed in the simulations in next section. The cross section area corresponding to the pressurized fluid should also be maximized according to the design principles, which implies a minimum partition wall thickness of 400 μm. This principle also implies that cross section deformation is also desirable, which however can be limited by PWS. Thus, the contributions of PWS and LCRS need to be matched to achieve the desired performance.

Referring now to FIG. 10, a system incorporating the manipulator 910 shown in FIGS. 9c and 9d is illustrated schematically.

The system is designated generally by the reference numeral 920 and comprises three pressure regulators 930, 940, 950. The three pressure regulators 930, 940, 950 are sealingly connected to the manipulator 910 by means of tubes 932, 942 and 952 respectively. Each of the tubes 932, 942, 952 feeds into one of the chambers 912.

The pressure in the chambers 912 may be varied individually in order to cause appropriate movement of the manipulator 910.

Compromise in Optimal Design Parameters

The optimal values of LCRS and PWS depend on the maximum pressure, denoted by pₘₐₓ, as well as the outer wall characteristics. Increasing the LCRS improves S₁/S₂, and thus the design principles indicate that it increases performance, as qualitatively shown in FIG. 10a. However, it requires a high PWS to prevent buckling, and therefore cross section deformation can be compromised, which can reduce performance. Conversely, lower PWS facilitates cross section deformation, which according to the design principles is desirable, leading to higher initial deflections and higher performance at lower pressures, as qualitatively shown in FIG. 11a. However, the maximum LCRS is then limited, which can reduce performance at higher pressures. A compromise is therefore necessary, which depends on pₘₐₓ. The performance of designs optimized for different pₘₐₓ is qualitatively illustrated in FIG. 11b, elucidating the fact that the optimal values of the parameters must be selected for the operating pressure in each scenario.

Since cross-sectional deformation is limited by the outer wall, all designs become equivalent in terms of cross section once full cross-sectional deformation is reached.

On the other hand, maximal PWS enables higher LCRS and therefore higher performance. In addition, high PWS also contributes to the longitudinal stiffness of wall 4, increasing S₁/S₂ and thereby leading to better performance. Thus, the optimization of the design involves selecting the maximum PWS that enables reaching full cross section deformation at Amax, and then the maximum LCRS to minimize tension at the outer wall during operation of the device while avoiding buckling. These parameters need to be optimized for each specific scenario. FE simulations were developed in this work for the optimization in the prototypical scenario. The specific simulations, the optimization process, and results obtained are reported in the next section.

It should be noted that, in the design selected, the partition wall stiffness serves to prevent buckling before full cross section deformation. After reaching full cross section deformation, this cross section remains practically constant despite further increases in pressure, and buckling does not occur since the contribution of the contracting effect is practically completed. Thus, designs with different PWS become equivalent once they reach full cross section deformation provided that the rest of the design is equal and that the contribution of the partition walls to the longitudinal stiffness is relatively low.

FE Simulations

FE simulations were developed in order to optimize the design parameters for the device in the prototypical scenario, and verify the design principles extracted in the previous subsections.

Evaluation Criteria

The criteria to evaluate the performance of the soft robotic manipulator 910 deserve consideration. The design objective in this prototypical scenario is to maximize the lateral force at a deflection near 20 degrees for a given pₘₐₓ. Thus, the performance is evaluated by measuring the normal force applied onto a prismatic block 960 positioned as shown in FIG. 12, with frictionless contact. This corresponds to an approximate deflection near 20 degrees of the manipulator at initial contact, and an interaction that is normal to the rigid block and approximately lateral on the soft robotic manipulator. It should be noted that the deflection at initial contact is somewhat lower than 20 degrees. This is intentional since the relative rotation between the ends of the manipulator varies with pressure even after contact, which implies that the distance between the center of the distal end of the device and the block changes even after contact. Since deflection is measured based on the position of the centers of the manipulator's ends, this varies at different pressures during contact. Thus, the rigid block is specifically positioned so that deflection is near 20 degrees for the range of pressures of interest.

This configuration selected for the simulations is also a representative of the typical operation of soft robotic manipulators. The design principles were shown to be independent of maximum pressure and deflection. Thus, the FE simulations conducted in this configuration also serve to verify some of the design principles derived in the previous sections.

Parameter Optimization

The objective of the optimization of the LCRS and PWS is to obtain both maximum PWS while reaching full cross section deformation at pₘₐₓ and minimal outer wall tension in the operation range of the device, while preventing buckling due to compression of the outer wall. This maximizes the forces that can be supported at pₘₐₓ and enables operation at low deflection.

The procedure to determine the optimal values of LCRS and PWS is as follows. First, PWS is selected to obtain full cross-sectional deformation at pₘₐₓ for a generic LCRS. This is achieved by conducting quasistatic simulations for a set of values of PWS with regular stiffness increments while maintaining constant material properties elsewhere. The simulations are executed using a gradual increase in pressure until a practically full cross-sectional deformation, which here is specified by the central rod reaching 70% of the radius, and the corresponding pressure is recorded. The PWS of the design that achieves practically full cross-sectional deformation at a pressure closest to pₘₐₓ is selected. Then, for the optimal PWS, the LCRS to achieve minimal outer wall tension is determined. This is done by conducting simulations with the optimal PWS and gradually increasing value for LCRS, starting with a stiffness corresponding to that of DragonSkin 10, until the outer wall stiffness is minimal. The LCRS that reaches minimal outer wall stiffness without buckling, together with the PWS to provide practically full cross section deformation at pₘₐₓ, constitute the optimal design. It should be noted that this optimization process of determining the PWS first independently of the LCRS is possible since cross-sectional deformation is relatively independent of the value of LCRS.

It should also be noted that rupture of the partition walls due to excessive strain is not considered since the maximum cross-sectional deformation is limited by the outer wall. The maximum possible extension of the partition walls is approximately double their initial length, which is significantly below the failure limit of typical rubbers. Similarly, extension of the central rod is typically lower than double the initial length, which is also below the failure limit. Thus, the PWS and LCRS are varied freely.

Simulation Implementation

The simulations were implemented using AbagusiStandard—Simulia™, Dassaut Systerries (Velizy-Villacoublay, France). The simulation set up involves the soft robotic manipulator and a rigid block situated as shown in FIG. 12. The geometry of the soft robotic manipulator is a 6 mm diameter cylinder, with a constant cross section as shown in FIG. 14a, a solid end cap of 1 mm thickness, and a total length of 31 mm.

The material of the outer wall was modeled as an incompressible, hyperelastic material with a Neo-Hookean constitutive law with c₁₀=42500 Pa and D=0, following [33]. The constitutive behavior of the material of the partition walls was also approximated with an incompressible Neo-Hookean law, and the different values of the PWS were selected by varying the parameter c₁₀, with values ranging between c₁₀=42500 Pa and c₁₀=425000 Pa at regular increments of 42500 Pa. Similarly, the material of the central rod was also approximated with an incompressible Neo-Hookean law, with a c₁₀ that was modified to vary LCRS. The bending stiffness of the central rod was not relevant at the stiffness values of interest since this was sufficiently thin. Finally, the fibers were modeled as circular beams of 10 μm diameter made of a material with a Young's modulus of 51 GPa, and a Poisson ratio of 0.36, which is representative of Kevlar.

An encastre boundary condition was imposed at one end of the manipulator, and another encastre was defined at one point of the rigid block. The contact between the manipulator and the rigid block was modeled as frictionless. The contact force was measured as the force applied by the manipulator on the rigid block.

The force corresponding to wall 6, T₂, was measured as the aggregated tension force over the outer wall of the device in a free body cut corresponding to the cross section indicated in FIG. 13. This is due to the fact that the outer wall in this 3D design provides the equivalent function as wall 6 in the analytical derivation. The mesh was maintained constant when varying material properties in the different simulations, and mesh convergence testing was conducted to ensure that the analysis was not affected by the characteristics of the mesh.

Simulation Results

The results of the simulations provide the deformation of the device and the force it applies on the rigid block as a function of pressure, as illustrated in FIG. 13 for a representative simulation. These results serve both to determine the optimal design parameters of the device in the prototypical scenario, and to verify some of the design principles.

Design Optimization Results

In terms of optimal parameters, the results of varying PWS for constant LCRS show that partition walls made of a material with c₁₀=127500 Pa yield practically full cross-sectional deformation at pₘₐₓ, as shown in FIG. 14a. This cross section corresponds to the section marked in orange in FIG. 13, which is representative of the cross-sectional deformation along the device. Thus, the optimal PWS in this scenario corresponds to c₁₀, =127500 Pa since it is the highest PWS that reaches practically full cross section deformation at pₘₐₓ.

The results of increasing LCRS for the optimal PWS are shown in FIG. 15. As can be seen, the performance improves with increasing values of LCRS. At an LCRS of c₁₀=425 MPa, the tension at the outer wall becomes zero and even slightly negative during operation, as can be seen in FIG. 16a, where the tension at the outer wall is plotted as a function of pressure for the different LCRS. Thus, the optimal LCRS corresponds to c₁₀=425 MPa since higher values of LCRS would involve compression stress at the outer wall, which could lead to structural instabilities such as buckling. This was confirmed by executing simulations at higher LCRS, which presented converge issues due to structural instabilities.

A design with partition walls made of a material with c₁₀=127500 Pa and central rod with c₁₀=425 MPa therefore represents optimal design in this scenario, together with the aforementioned geometry and the outer wall made of DragonSkin 10 with c₁₀=42500 Pa. The higher performance of the optimal design is predominantly due to two factors. First, it presents practically full cross section deformation at pₘₐₓ, and therefore it provides a high performance in terms of cross section as the area corresponding to the pressurized fluid is maximized and the majority of the stiffness is concentrated near the cross section contour corresponding to wall 4. Second, it has the highest LCRS, and therefore the force spent stretching the structure is minimized, particularly at the central rod which corresponds to wall 4, leading to a maximal contribution of pressure to support external forces. Interestingly, a relation can be observed between the reduction in tension at the outer wall, shown in FIG. 16a, and the improvement in performance due to the increase in LCRS, shown in FIG. 14, where the magnitude of the improvement in performance between two designs is directly related to the magnitude of the reduction in outer wall tension.

The results of the simulations show that buckling of the outer wall does not occur, as can be seen in FIG. 13. The results also indicate that any bulging of the outer wall between the circumferential fibers is minor, as can also be seen in FIG. 13, which corresponds to a strain at the outer wall that is maintained significantly below the failure limit of the rubber. Thus, the wall performs as desired, withstanding the pressure applied without excessive wall thickness.

This outer wall behaves similarly to a pleated structure, presenting longitudinal extension with only minimal radial expansion between the fibers. Thus, this design with the soft outer wall and circumferential fibers is mostly equivalent to a previously mentioned design with a pleated structure and circumferential fibers, and the study developed here can be generally extrapolated due to the similar structural behavior.

The results of the simulations also confirm that the tension at the inner rod is relevant and significant. Observing the end cap, as shown in FIG. 13, a depression can be noted, which is caused by the inner rod in tension.

Performance Comparison

The performance of the design obtained in this work with optimal parameters was also compared with that of the FMA, since it is as well-established design, and is representative of some of the highest performing soft robotic manipulators that can meet the requirements of the scenario defined in this work. DragonSkin 10 was selected as the material for the FMA in order to compare both designs in equivalent conditions. The results of lateral force as a function of pressure are shown in FIG. 14. As can be seen, the design obtained here provides a higher force at pₘₐₓ. The results also show that, at lower pressures, the FMA presents a somewhat higher performance, primarily due to the softer partition walls that enable larger cross-sectional deformation at lower pressure, confirming that a design must be optimized for a specific pressure.

Alternative Materials

The design obtained in this work can be fabricated using readily available silicones for the outer wall, partition walls, and fibers. However, the hyperelastic material selected for the central rod can be difficult to obtain in practice as it presents a stiffness significantly higher than that of standard rubbers. In order to consider more realistic materials, equivalent simulations were conducted using elastic material properties for the central rod, with Young moduli between E=10⁸ Pa and E=10¹⁰ Pa, which are representative of cotton or wool threads. The results are shown in FIG. 15, together with the previous results for hyperelastic central rod. As can be seen, the performance of designs with central rods made of stiff, elastic materials are equivalent to those with hyperelastic materials. Thus, the design can be fabricated by using readily available materials such as textile threads as the central rod.

Principles and Operation Verification Results

The results of the simulations also serve to verify two of the most relevant design principles. In addition, they can be used to confirm that the operation of the device is as predicted.

The performance of different designs with varying PWS and constant LCRS and material properties elsewhere is plotted in FIG. 15a as a function of pressure. The plots indicate that lower PWS increases the lateral force that the device can apply, and reduces the pressure required to attain an initial deflection. These results agree with the behavior predicted based on this analysis in this work, shown in FIG. 11a. Thus, the results confirm that cross-sectional deformation is desirable to improve performance. Equivalently, the results verify that maximizing the area of the cross section corresponding to the pressurized fluid is desirable to maximize the force of soft robotic manipulators. This contrasts with some of the designs in the literature [19], and shows that, unless additional constraints are present, such as those exposed in subsection 4.6, the exploitation of cross-sectional deformation can yield designs with improved performance.

The results of increasing values of LCRS with a constant design elsewhere, shown in FIG. 14 for both hyperelastic and elastic central rods, confirm that increasing LCRS leads to higher force in general. These results also agree with the predicted trends, shown in FIG. 11b. This verifies another of the design principles, namely that high LCRS is desirable to maximize the performance or, equivalently, that maximal S₁/S₂ is desirable to maximize the force that can be applied, provided that it does not lead to buckling of wall 6. It should be noted that the result of the simulation with a central rod stiffness of c₁₀=4.25 MPa does not reach the full pressure. This is due to the fact that the simulation did not converge at pressures above 5.7 psi since some mesh elements presented excessive distortion. Nonetheless, the plot elucidates the trends of interest.

Finally, the results of tension at the outer wall for different LCRS, shown in FIG. 16a, confirm that increasing LOPS leads to lower values of overall tension at the outer wall. Thus, these results confirm that the performance improves as less force is spent stretching the outer wall. In particular, for the optimal LCRS, the results in FIG. 16a show that the tension at the outer wall becomes zero and even to a slight extent negative, which indicates that the objective of the optimization in terms of minimizing outer wall tension is achieved. Moreover, the results on outer wall tension confirm that the contracting operation is effective, particularly at low deflections, where the tension at the equivalent of wall 6 becomes practically zero. At larger deflections, the contribution of the contracting operation is significantly reduced, since the protrusion is limited by the outer wall and cannot increase further. Then, the extending operation becomes relevant, which involves some inevitable tension at the outer wall, but provides a high overall performance.

Embodiments of the invention have application in many areas of technology.

One such area is the field of jet engine inspection in which a soft robotic manipulator can be inserted into an engine while it is on-wing.

In such applications, the manipulator should be attachable to the tip of a borescope type tool in which a camera normally forming part of the tool has been removed leaving an open lumen of approximately 2 mm diameter.

Alternatively, the manipulator could also be attachable to another flexible device that has an open lumen of about 1 to 2 mm, or the equivalent room to accommodate the microtube supplying pressure to the manipulator.

In this application, the manipulator is ideally formed from two segments, each of which segments is capable of bending in any direction in three dimensions with reflections of up to 90°. In this application the maximum diameter of the outer wall of the manipulator will be approximately 15 mm and the manipulator will have a length of 90 mm in order that it is compatible with the body of the borescope.

In this application, any pressure value can be used since a jet engine is robust and therefore issues such as the health of a patient are not relevant.

This application corresponds to the case of maximum pressure determined by the design in terms of outer wall thickness mentioned above. The design method for this case is shown by way of example in the following analysis and derivation.

Since the robot is able to operate at higher pressure than is the case for the manipulator described above and with reference to FIGS. 9a to 9c, higher forces may be used which can be desirable. However, higher pressure also requires a thicker outer wall to prevent the robot from bursting. This thicker outer wall introduces bending stiffness and reduces the region of the cross-section that corresponds to pressurised chambers, both of which reduce the force of the device. This means a compromise must be found.

The design principles for this case are as described hereinabove with regards to methods according to embodiments of the invention. The difference in this case is that the thickness of the outer wall is to be selected based on the comprise described above which adds two more design principles:
1. a thicker outer wall allows higher pressures, which is desirable as it leads to higher forces; and
2. a thicker outer wall increases bending stiffness and reduces chamber area which is undesirable as it reduces the resultant force of the design.

In accordance with embodiments of the invention therefore it is first necessary to consider that each robot segment must bend up to significant deflections, and to support maximum at any deflection.

This means that the best design should be a combination of extending device and contracting device, as in the examples set out above.

Since the device needs to bend in any direction in three-dimensions, the design needs to have at least three partition walls that define three chambers. The number of partition walls is then selected to be three in order that the cross-section region corresponds to the pressurised fluid should be maximised and the bending stiffness of the manipulator should be minimised.

Embodiments of the invention are then applied to finalise the design. In this case the outer diameter of the manipulator will be approximately 15 mm and the thickness of the outer wall will be approximately 2.5 mm. The thickness of the partition walls will be 1.5 mm and the rod will be made of silicon with an embedded fibre made of a material having wool-like properties with a diameter of 0.1 mm and a Young's Modulus of 4 e¹⁰.

Another application of a manipulator in accordance with embodiments of the invention is another medical application requiring bending in a plane between −120° and +120c with a maximum pressure limited to 15 psi, a length of 25 mm and a cross-section limited to a square of 4 mm in length.

Again, methods according to embodiments of the invention may be used to determine an optimum design.

The possible values of partition wall stiffness must be divided in two cases. In some designs the partition walls can be made of the same material as the outer wall, whereas in other designs the partition walls can be made of a different material from the outer wall.

If the partition walls are made of the same material as the outer wall, the stiffness of the partition walls and outer wall is determined by their thickness. Typical values of the partition wall thickness and outer wall thickness are between ¼ of the total diameter of the device and 1/20 of the total diameter. The material of both partition walls and outer wall can have a stiffness between that of Ecoflex 00 30 (https://www.smooth-on.com/productslecoflex-00-30/) and that of Smooth-sil 950 (https://www.smooth-on.com/productsismooth-sil-950/). Unfortunately, the stiffness of these silicones is not measured using Young's modulus, but using shore hardness. In general, we could say that the partition walls and outer walls cam be made of rubber with a Shore hardness between OO 30 and OO 90, using the Shore OO scale, or similarly between A 0 and A 60, using the Shore A scale.

If the partition walls are made of a different material from the outer wall, then their stiffness can be expressed relative to the stiffness of the outer wall. In this case, we could say the outer wall can be made of materials with a Shore hardness between OO 30 and OO 90, using the Shore OO scale, or similarly between A 0 and A 60, using the Shore A scale. The partition walls then can have a stiffness between ¼ of the stiffness of the outer wall and 2 times the stiffness of the outer wall.

1. A soft robotic manipulator adapted to be activated by a pressurised fluid having a first end, a second end, an outer wall and an axis, and comprising a plurality of segments extending co-axially along the manipulator, such that the outer wall of each segment forms part of the outer wall of the manipulator, each segment having a first end and a second end and an outer wall and further comprising a plurality of chambers contained within the outer wall, each of which chambers extends from the first end to the second end, wherein each manipulator segment further comprises a central element extending along the axis of the manipulator segment, and a plurality of partition walls extending from the central element to the outer wall, the chambers being defined by the partition walls and the outer wall, wherein the outer wall of the manipulator comprises the outer wall of each segment.

2. The manipulator of claim 1 wherein the outer wall has a substantially circular or rectangular cross-section.

3. The manipulator of claim 1 wherein the central element is an inextendible rod or an extendible sheet.

4. The manipulator of claim 1 wherein the partition walls are formed from a material having a material having a Young's modulus in the range 10⁴ to 10⁹ and/or the partition walls have a thickness of between 1/20 and ¼ of the total diameter of the manipulator.

5. The manipulator of claim 1 wherein the outer wall is made of a material having a Young's modulus in the range 10⁴ to 10⁹ and/or a thickness of between 1/20 and ¼ of the total diameter of the manipulator.

6. The manipulator of claim 1 wherein the outer wall includes a tubular structure having notches, and optionally the tubular structure is formed from a metallic material, optionally nitinol.

7. The manipulator of claim 1 wherein the outer wall has a pleated structure.

8. The manipulator of claim 1 wherein the central element comprises an extendible rod with a stiffness equivalent to that of a fibre with a 0.1 mm diameter and a Young's modulus between 1 e⁷ to 1 e¹⁰.

9. The manipulator of claim 1 wherein the central element is inextendible and is made of parts with negligible bending stiffness and rigid parts, whereby when a differential pressure is applied in the chambers, the increase in volume in the pressurised chambers is maximised for a given increase in bending, and thus the force of the device is maximised.

10. The manipulator of claim 1 wherein the total cross section of the device occupies all available space in a selected application.

11. The manipulator of claim 1 wherein the central element acts as a stiff wall in extending devices and as the protruding wall in contracting devices, whereby the resulting device combines extending and contracting operation into a fully integrated device.

12. A method of designing a soft robotic manipulator with fluidic actuation comprising the steps of:
i. identifying the conditions under which the manipulator will be operated
ii. identifying the requirements to be fulfilled by the manipulator;
iii. based on i and ii, selecting an extending, contracting or combination type manipulator;
iv. depending on the category of manipulator selected, and the conditions and requirements to be met, create a preliminary cross-section design;
v. then consider the optimal stiffness design required in light of ii, iii, and iv;
vi. repeat steps iv and v as necessary in order to optimize the design;
vii. identify the layout of the manipulator
viii. optimize the design.

13. The method of claim 12 wherein step i comprises identifying the spatial constraints in the environment in which the manipulator will operate.

14. The method of claim 12 in which step ii comprises identifying a desired deflection of the manipulator during operation of the manipulator.

15. The method of claim 12 wherein step iii comprises selecting a contracting device if the desired deflection is relatively low and there is sufficient space to allow the manipulator to bulge during contraction.

16. The method of claim 12 wherein step iii comprises selecting a extending device if the desired deflection is high, or the available space is limited.

17. The method of claim 12 wherein step iii comprises selecting a combination of extending and contracting if the desired deflection varies across a broad range of values.

18. The method of claim 12 comprising selecting both an extending device and a contracting device and then determining which device has optimal performance.

19. The method of claim 12 wherein step iv comprises selecting a cross section for the device to occupy a maximum proportion of the space available.

20. The method of claim 19 further comprising the further step of designing a preliminary cross sectional geometry including determining the size of the walls of the device.

21. The method of claim 12 wherein step viii comprises using FE simulations to optimise the design.

22. A soft robotic manipulator designed using a method according to claim 12.
